(19) United States
(12) Patent Application Publication
(10) Pub. No.: US 2015/0381923 A1
(43) Pub. Date: Dec. 31, 2015

(54) METHODS AND SYSTEMS FOR ADJUSTING A PLAY LENGTH OF A MEDIA ASSET BASED USER ACTIONS

(71) Applicant: United Video Properties, Inc., Santa Clara, CA (US)

(72) Inventors: Vanessa Wickenkamp, Elmhurst, IL (US); Andrew Fundament, Arlington Heights, IL (US); Douglas J. Seyller, Lisle, IL (US); Andy Dustin, Palos Hills, IL (US); James Cornell, Chicago, IL (US); Melissa Bradley, Mt. Prospect, IL (US)

(21) Appl. No.: 14/317,497

(22) Filed: Jun. 27, 2014

(51) Int. Cl. H04N 5/783; A61B 19/00; G11B 27/36; H04N 5/91; G11B 27/10

(52) U.S. Cl. CPC H04N 5/783; H04N 5/91; G11B 27/102; G11B 27/36; A61B 19/00

(57) ABSTRACT

Methods and systems are disclosed herein for automatically adjusting a play length of a media asset based on actions of a user. For example, a media guidance application may select a media asset for presentation while a user brushes his teeth. Based on the progress of the teeth cleaning, the play length of the media asset may be adjusted.

### BACKGROUND

Users commonly access playlists of media content at particular time periods in a day. For example, after waking up in the morning, users may listen to music as they perform one or more activities (e.g., brush teeth, brush hair, shower, eat breakfast, etc.). Typically, a user is free to modify (e.g., pause, fast-forward, skip, etc.) the playback of the playlist. However, while a user may freely modify the playback of the media playlist, modifying the playback (e.g., changing from one program to another) is time-consuming and may thus interfere with and/or delay the performance of the activities.

### SUMMARY

Accordingly, methods and systems are disclosed herein for automatically adjusting media playlists, and media assets within those playlists, based on one or more activities performed by a user. For example, a media guidance application may select media assets for inclusion in a media playlist that correspond to particular activities. Moreover, the media playlist may be structured such that the selected media assets are automatically presented while the user is performing the particular activities.

For example, a media guidance application may determine that each morning a user wakes up, takes a shower, brushes his or her teeth, and gets dressed. Moreover, the media guidance application may identify particular media assets to accompany each of these activities. These identified media assets may have subject matter, play lengths, output types, etc. that correspond to the activity for which each accompanies. The media guidance application may then include these media assets into a media playlist such that each media asset is timed (e.g., begins when a user begins the respective activity and ends when the user ends the respective activity).

Moreover, the media guidance application may monitor the progress of the user during each activity. For example, if a user is taking an unusually long time to shower, the media guidance application may modify the play length of the media asset that accompanies that activity such that it continues to be presented while the user showers. Alternatively or additionally, the media guidance application may modify the play length of a media asset that accompanies an activity that follows the current activity (e.g., in order to keep a user on schedule). Alternatively or additionally, the media guidance application may provide user progress feedback while the user is performing the first activity. For example, the media guidance application may present a time indicating how much time remains to complete the activity.

In some aspects, a media guidance application may determine a first activity and a second activity to be performed by a user in series. For example, the media guidance application may receive a user input indicating that during a certain time period (e.g., seven o'clock to eight o'clock) the user performs three different activities in series. Alternatively or additionally, the media guidance application may passively monitor a user and generate a user profile of typical habits or routines of the user in order to determine what activities, and when, will be performed.

The media guidance application may then determine a first amount of time required to perform the first activity and a second amount of time to perform the second activity. For example, the media guidance application may cross-reference a database that lists approximate times for different activities to determine an approximate time for each of the first and second activities.

The media guidance application may then select a first media asset to present to the user while the user is performing the first activity and a second media asset to present to the user while the user is performing the second activity, in which a play length of the first media asset corresponds to the first amount of time and a play length of the second media asset corresponds to the second amount of time. For example, if the first activity (e.g., eating breakfast) requires ten minutes to perform, the media guidance application may select a media asset (e.g., a news clip) that last for ten minutes. Alternatively, the media guidance application may select several media assets that, when combined, last for ten minutes.

In response to determining that the first activity has been completed, the media guidance application may compare the first amount of time to an actual amount of time required to perform the first activity. For example, the media guidance application may monitor user progress corresponding to the performance of the first activity to determine when the first activity has been completed.

In response to detecting a discrepancy between the first amount of time and the actual amount of time, the media guidance application may adjust the play length of the second media asset to rectify the discrepancy. For example, if a user completed the first activity earlier than scheduled, the media guidance application may give the user more time to perform the second activity. Accordingly, the media guidance application may adjust the play length of the second media asset by extending the play length of the second media asset to include additional content. In another example, if a user completed the first activity later than scheduled, the media guidance application may give the user less time to perform the second activity. Accordingly, the media guidance application may adjust the play length of the second media asset by decreasing the play length of the media asset by cropping a portion of media asset.

In some embodiments, the media guidance application may receive user input indicating criteria for select media assets. For example, the media guidance application may receive a user input selecting a subject matter for the first media asset and the second media asset and then search a media content source for media assets corresponding to the subject matter. In another example, the media guidance application may determine a first output format (e.g., video, audio, textual, etc.) for the first media asset and a second output format for the second media asset based on the activity being performed (e.g., whether or not a particular activity is conducive to a user consuming a video simultaneously). In response, the media guidance application may search a media content source for media assets corresponding to the first output format and the second output format.

In some embodiments, the media guidance application may reward a user for completing one or more activities early. For example, the media guidance application may provide the user with additional content, an increased score, or a product offering in response to determining that the user has completed one or more activities earlier than scheduled.

In some aspects, the media guidance application may detect that a user has begun a teeth cleaning using a user device (e.g., an electric tooth brush). For example, the media guidance application may monitor a user to determine when the user uses a specific user device, or the media guidance application may receive information that is transmitted from the user device that indicates that a user has begun an activity. In response to detecting that the user has begun the teeth cleaning, the media guidance application may determine an initial amount of time remaining in the teeth cleaning based on an initial condition. For example, the user device may determine a total surface area that needs to be cleaned or an amount of dental plaque that needs to be removed and calculate an amount of time remaining in the teeth cleaning based that determination. Additionally or alternatively, the media guidance application may access a user profile associated with the user that indicates a typical length of time required for the user to perform the teeth cleaning.

The media guidance application may then retrieve a media asset with a play length that corresponds to the initial amount of time. For example, after determining how long a user is likely to take to perform the teeth cleaning, the media guidance application may search for a media asset with a play length that matches the determined length of time.

The media guidance application may then determine a current amount of time remaining in the teeth cleaning based on a current condition. For example, the user device may periodically determine the total surface area of the teeth that still needs to be cleaned or determine an amount of dental plaque that still needs to be removed and calculate a new amount of time remaining in the teeth cleaning based the latest determination.

The media guidance application may compare the current amount of time to the remaining play length. For example, the media guidance application may determine whether the initial amount of time corresponds to the current amount of time in order to determine whether or not the user is on schedule.

The media guidance application may then adjust the play length of the media asset to rectify the discrepancy. For example, in response to determining that the teeth cleaning will take longer (or shorter) than initially expected, the media guidance application may modify the length of time it takes for the user to consume a media asset that accompanies the activity. For example, the media guidance application may extend the play length of the media asset by including additional content. In another example, the media guidance application may extend the play length of the media asset by reducing a playback rate associated with the media asset. In another example, the media guidance application may decrease the play length of the media asset by advancing a current point of playback in the media asset to a later point of playback in the media asset. In yet another example, the media guidance application may decrease the play length of the media asset by cropping a portion of media asset.

In some embodiments, the media guidance application may additionally provide user progress feedback to the user while the user is performing the teeth cleaning. For example, the media guidance application may indicate how much time remains for the scheduled activity. Alternatively or additionally, the media guidance application may provide instructions associated with the teeth cleaning. For example, the media guidance application may instruct the user about particular teeth that need to be cleaned.

It should be noted that the systems and/or methods described above may be applied to, or used in accordance with, other systems, methods and/or apparatuses.

### BRIEF DESCRIPTION OF THE DRAWINGS

The above and other objects and advantages of the disclosure will be apparent upon consideration of the following detailed description, taken in conjunction with the accompanying drawings, in which like reference characters refer to like parts throughout, and in which:

FIG. 1 shows an illustrative example of a display screen generated by a media guidance application that lists a plurality of media listing in accordance with some embodiments of the disclosure;

FIG. 2 shows another illustrative example of a display screen generated by a media guidance application for use in generating for display media assets to accompany an activity performed by a user in accordance with some embodiments of the disclosure;

FIG. 3 is a block diagram of an illustrative user equipment device in accordance with some embodiments of the disclosure;

FIG. 4 is a block diagram of an illustrative media system in accordance with some embodiments of the disclosure;

FIG. 5 is a illustration of a user device linked to the presentation of a media asset in accordance with some embodiments of the disclosure;

FIG. 6 is a flowchart of illustrative steps for adjusting the play length of a media asset associated with a current activity based on the length of time required to perform a previous activity in accordance with some embodiments of the disclosure;

FIG. 7 is a flowchart of illustrative steps for adjusting the play length of a media asset based on a user's progress in an activity in accordance with some embodiments of the disclosure;

FIG. 8 is a flowchart of illustrative steps for searching for media assets based on particular criteria in accordance with some embodiments of the disclosure; and

FIG. 9 is a flowchart of illustrative steps for adjusting a playlist based on the length of time required to perform an activity in accordance with some embodiments of the disclosure.

### DETAILED DESCRIPTION OF THE DRAWINGS

Methods and systems are disclosed herein for automatically adjusting media playlists, and media assets within those playlists, based on one or more activities performed by a user. For example, a media guidance application may select media assets for inclusion in a media playlist that correspond to particular activities. Moreover, the media playlist may be structured such that the select media assets are automatically presented while the user is performing the particular activities.

As referred to herein, “a media guidance application,” “interactive guidance application,” or “guidance application” relates to an interface that allows users to efficiently view, select, and/or navigate between content selections and easily identify content that they may desire. The media guidance application and/or any instructions for performing any of the embodiments discussed herein may be encoded on computer-readable media. Computer-readable media includes any media capable of storing data. The computer-readable media may be transitory, including, but not limited to, propagating electrical or electromagnetic signals, or may be non-transitory including, but not limited to, volatile and nonvolatile computer memory or storage devices such as a hard disk, floppy disk, USB drive, DVD, CD, media cards, register memory, processor caches, Random Access Memory (“RAM”), etc.

Media guidance applications may take various forms depending on the content for which they provide guidance. One typical type of media guidance application is an interactive television program guide. Interactive television program guides (sometimes referred to as electronic program guides) are well-known guidance applications that, among other things, allow users to navigate among and locate many types of content or media assets. Interactive media guidance applications may generate graphical user interface screens that enable a user to navigate among, locate and select content.

In another example, a media guidance application may automatically present media content and/or adjust that media content. Furthermore, in some embodiments, the media guidance application may present and/or modify the media content based on actions of a user. For example, a media guidance application may determine that each morning a user wakes up, takes a shower, brushes his or her teeth, and gets dressed. Moreover, the media guidance application may identify particular media assets to accompany each of these activities. These identified media assets may have subject matter, play lengths, output types, etc. that correspond to the activity for which each accompanies. The media guidance application may then include these media assets into a media playlist such that each media asset is timed (e.g., begins when a user begins the respective activity and ends when the user ends the respective activity).

Moreover, the media guidance application may monitor the progress of the user during each activity. For example, if a user is taking an unusually long time to shower, the media guidance application may modify the play length of the media asset that accompanies that activity such that it continues to be presented while the user showers. Alternatively or additionally, the media guidance application may modify the play length of a media asset that accompanies an activity that follows the current activity (e.g., in order to keep a user on schedule). Alternatively or additionally, the media guidance application may provide user progress feedback while the user is performing the first activity. For example, the media guidance application may present a time indicating how much time remains to complete the activity.

As referred to herein, a “play length” of a media asset is the amount of time required to consume the media asset. For example, if the media asset is an audio or video media asset, the play length of the media asset may correspond to the run-time of the audio or video media asset. If the media asset is a video game or textual media asset (e.g., a media asset without a fixed run-time), the play length may correspond to a length of time that it take for the user (or an average user) to complete or read the media asset.

As referred to herein, the terms “media asset” and “content” should be understood to mean an electronically consumable user asset, such as television programming, as well as pay-per-view programs, on-demand programs (as in video-on-demand (VOD) systems), Internet content (e.g., streaming content, downloadable content, Webcasts, etc.), video clips, audio, content information, pictures, rotating images, documents, playlists, websites, articles, books, electronic books, blogs, advertisements, chat sessions, social media, applications, games, and/or any other media or multimedia and/or combination of the same. Guidance applications also allow users to navigate among and locate content. As referred to herein, the term “multimedia” should be understood to mean content that utilizes at least two different content forms described above, for example, text, audio, images, video, or interactivity content forms. Content may be recorded, played, displayed or accessed by user equipment devices, but can also be part of a live performance.

In some embodiments, a media guidance application may determine a first activity and a second activity to be performed by a user in series. For example, the media guidance application may receive a user input indicating that during a certain time period (e.g., seven o'clock to eight o'clock) the user performs three different activities in series. Alternatively or additionally, the media guidance application may passively monitor a user and generate a user profile of typical habits or routines of the user in order to determine what activities, and what time they will be performed.

As referred to herein, an “activity” refers to a specified pursuit in which a user partakes that requires movement of the body. For example, the activity may be a physical activity, which is an activity that requires bodily movement produced by skeletal muscles that requires energy expenditure. The activity may also be a social activity, which is an activity that involves more than a single person. In some embodiments the activity may accompany the consumption of a media asset. For example, activities such as attending a movie, eating dinner, riding in a taxi, driving a car, etc. may all be accompanied by the presentation of a media asset (e.g., a movie, a song, a game, etc.).

In some embodiments, activities may be performed in series. For example, the activities may be performed consecutively and/or in a particular order. Alternatively, activities may be performed non-consecutively and/or in any order. In some embodiments, the activities may correspond to a particular time of day or a period of time.

The media guidance application may then determine a first amount of time required to perform the first activity and a second amount of time to perform the second activity. For example, the media guidance application may cross-reference a database that lists approximate times for different activities to determine an approximate time for each of the first and second activities.

In some embodiments, the media guidance application may base the amount of time on prior user behavior. For example, the media guidance application may monitor the user's (or other users') past history to determine average times for each activity. Alternatively or additionally, the media guidance application may determine an amount of time for each activity based on other scheduled activities. For example, if the media guidance application determines that a user needs to leave for work at a particular time the media guidance application may recommend or automatically generate a schedule that assigns specific amounts of time to various activities. In another example, the media guidance application may receive a user input (e.g., from a parent) indicating a particular schedule that another user (e.g., a child) must abide by.

The media guidance application may then select a first media asset to present to the user while the user is performing the first activity and a second media asset to present to the user while the user is performing the second activity, in which a play length of the first media asset corresponds to the first amount of time and a play length of the second media asset corresponds to the second amount of time. For example, if the first activity (e.g., eating breakfast) requires ten minutes to perform, the media guidance application may select a media asset (e.g., a news clip) that last for ten minutes. Alternatively, the media guidance application may select several media assets that when combined last for ten minutes.

It should be noted that any embodiment discussed herein as applied to presenting, selecting, etc. a media asset to accompany an activity may also be applied to an embodiment featuring a portion of a media asset or several media assets (e.g., a playlist) and vice versa. For example, in response to determining that several activities are scheduled to be performed by a user in series, the media guidance application may separate a single media asset (e.g., a movie) into several parts (e.g., each part corresponding to one or more activities). In another example, the media guidance application may select several media assets (e.g., in a playlist) for presentation during a single activity.

In some embodiments, the media guidance application may receive user input indicating (or may determine automatically) criteria for selecting media assets. For example, the media guidance application may receive a user input selecting a subject matter for the first media asset and the second media asset and then search a media content source for media assets corresponding to the subject matter. Alternatively, the media guidance application may access user preference contained in a user profile to determine media content that a user is likely to enjoy.

In another example, the media guidance application may determine a first output format (e.g., video, audio, textual, etc.) for the first media asset and a second output format for the second media asset based on the activity being performed (e.g., whether or not a particular activity is conducive to a user consuming a video simultaneously). In response, the media guidance application may search a media content source for media assets corresponding to the first output format and the second output format.

In some embodiments, the media guidance application may modify the media assets or present supplemental content with the media assets (e.g., overlaid on the media assets). For example, the media guidance application may provide user progress feedback to the user while the user is performing an activity. In some embodiments, the feedback may indicate an amount of time remaining for an activity, may encourage the user to perform the activity faster or slower, etc. For example, the media guidance application may count down how much time remains for the scheduled activity.

Alternatively or additionally, the media guidance application may provide instructions associated with the activity. For example, the media guidance application may instruct the user to “Remember to wash behind your ears” during a shower, or remind a user to floss when cleaning his or her teeth. Such reminders may be presented by a device a user (e.g., on a screen incorporated into or associated with the device or projected onto another surface) is using to perform the activity and/or a device presenting a media asset.

As referred to herein, the phrase “user equipment device,” “user equipment,” “user device,” “electronic device,” “electronic equipment,” “media equipment device,” or “media device” should be understood to mean any device for accessing the content or performing activities described herein, such as a television, a Smart TV, a set-top box, an integrated receiver decoder (IRD) for handling satellite television, a digital storage device, a digital media receiver (DMR), a digital media adapter (DMA), a streaming media device, a DVD player, a DVD recorder, a connected DVD, a local media server, a BLU-RAY player, a BLU-RAY recorder, a personal computer (PC), a laptop computer, a tablet computer, a WebTV box, a personal computer television (PC/TV), a PC media server, a PC media center, a hand-held computer, a stationary telephone, a personal digital assistant (PDA), a mobile telephone, a portable video player, a portable music player, a portable gaming machine, a smart phone, or any other television equipment, computing equipment, or wireless device, electric toothbrush, a wearable electronic device (e.g., glasses, wristbands, etc.), hairbrush, vacuum, mop, broom, washing machine, dishwasher, alarm clock and/or any other household appliance. In some embodiments, the user equipment device may have a front facing screen and a rear facing screen, multiple front screens, or multiple angled screens. In some embodiments, the user equipment device may have a front facing camera and/or a rear facing camera. On these user equipment devices, users may be able to navigate among and locate the same content available through a television. Consequently, media guidance may be available on these devices, as well. The guidance provided may be for content available only through a television, for content available only through one or more of other types of user equipment devices, or for content available both through a television and one or more of the other types of user equipment devices. The media guidance applications may be provided as on-line applications (i.e., provided on a web-site), or as stand-alone applications or clients on user equipment devices. Various devices and platforms that may implement media guidance applications are described in more detail below.

In response to determining that the first activity has been completed, the media guidance application may compare the first amount of time to an actual amount of time required to perform the first activity. For example, the media guidance application may monitor user progress corresponding to the performance of the first activity to determine when the first activity has been completed.

In some embodiments, the media guidance application may incorporate or have access to a detection module for use in determining whether or not a user has begun, is performing, or has completed an activity. As discussed below, the detection module may use a plurality of techniques to monitor the user and the actions of the user.

In some embodiments, the media guidance application may track the progress of the user through the use of one or more timing mechanisms. For example, an interval clock mechanism may compare a current amount of time remaining in an activity to a corresponding schedule, and present a particular media asset and/or adjust a media asset based on the comparison. The media guidance application may also use GPS tracking to determine whether or not a user is at a location of a scheduled activity at a correct time. If the user is not at the correct location, the media guidance application may adjust the media asset. For example, the media guidance application may determine if a user is ahead of or behind schedule based on his/her position in the household (e.g., in the bathroom as opposed to the kitchen). In response to determining that the user's progress deviates from the schedule, the media guidance application may adjust one or more media assets and/or the position of the media assets in a playlist.

In some embodiments, the media guidance application may implement a checkpoint system whereby the user “checks into” each activity. Upon “checking in” or “checking out,” the media guidance application adjusts one or more media assets. For example, upon beginning a first activity (e.g., as indicated by a user “checking into” the activity), the media guidance application may present a media asset corresponding to that activity.

In response to detecting a discrepancy between the first amount of time and the actual amount of time, the media guidance application may adjust the play length of the second media asset to rectify the discrepancy. For example, if a user completed the first activity earlier than scheduled, the media guidance application may give the user more time to perform the second activity. It should be noted that “adjusting” a play length of a media asset may include extending the play length of the second media asset to including additional content, decreasing the play length of the media asset by cropping a portion of the media asset, reducing a playback rate associated with the media asset, advancing a current point of playback in the media asset to a later point of playback in the media asset, pausing the playback of the media asset, and/or performing any fast-access playback operation on the media asset. Additionally or alternatively, the media guidance application may select another media asset (e.g., after an initial media asset has been completed) in response to determining a need to adjust a play length of a media asset. In some embodiments, the adjustments may be automatic or require a user instruction.

As referred to herein, the phrase “fast-access playback operations” should be understood to mean any media guidance application operation that pertains to playing back a non-linear media asset faster than normal playback speed or in a different order than the media asset is designed to be played, such as a fast-forward, rewind, skip, chapter selection, segment selection, skip segment, jump segment, next segment, previous segment, skip advertisement or commercial, next chapter, previous chapter or any other operation that does not play back the media asset at normal playback speed. The fast-access playback operation may be any playback operation that is not “play,” where the play operation plays back the media asset at normal playback speed.

It should be noted that any embodiment discussing adjusting a media asset may also be applied to adjusting a media playlist. For example, instead of cropping a media asset in order to shorten the play length of the media asset, the media guidance application may remove a media asset from the playlist in order to shorten the play length of the playlist. Additionally, in some embodiments, the media guidance application may adjust a media playlist by modifying the position of one media asset (e.g., relative to another media asset) in the playlist. For example, in response to determining that a user decided to perform one activity before another (e.g., in contrast to the normal routine of the user), the media guidance application may switch the positions of the media assets that accompanied those activities in the playlist.

In some embodiments, the media guidance application may reward a user for completing one or more activities early or longer than scheduled. For example, the media guidance application may provide the user with additional content, an increased score, or a product offering in response to determining that the user has completed one or more activities earlier (or later) than scheduled. For example, in order to encourage a user (e.g., a child) to get out of bed on time, the media guidance application may reward the user for performing an activity (e.g., waking up) prior to a scheduled time. Likewise, the media guidance application may reward the user if, after several activities (e.g., showering, teeth cleaning, etc.), the user is still ahead of schedule.

In some embodiments, the media guidance application may further adjust a media asset or playlist based on conditions related to an activity. For example, the media guidance application may adjust a media asset keyed to the activity of eating breakfast based on how much food the user has currently eaten. In another example, the media guidance application may detect that a user has begun brushing his or her teeth using a user device (e.g., an electric tooth brush). For example, the media guidance application may monitor a user to determine when the user uses a specific user device, or the media guidance application may receive information that is transmitted from the user device that indicates that a user has begun an activity.

In response to detecting that the user has begun the teeth cleaning, the media guidance application may determine an initial amount of time remaining in the teeth cleaning based on an initial condition. As used herein, a “condition” may be any quantitative or qualitative measure of the current circumstances surrounding the performance of an activity. For example, a condition may relate to a total surface area that needs to be cleaned or an amount of dental plaque that needs to be removed in order to complete an activity (e.g., teeth cleaning).

The media guidance application may use numerous techniques to determine a condition associated with an activity. For example, sensors attached to a user device (e.g., an electronic tooth brush) may monitor for one or more indicia of a condition. For example, the user device may be equipped with sensors for determining a level of dental plaque on the teeth of a user. Sensors associated with a user device are more fully discussed in Bates et al. U.S. patent application Ser. No. 13/820,551, filed Aug. 5, 2011 and Schultz U.S. patent application Ser. No. 11/555,115, filed Oct. 31, 2006, which are hereby incorporated by reference herein in their entireties.

The media guidance application may then retrieve a media asset with a play length that corresponds to the initial amount of time. For example, after determining how long a user is likely to take to perform the teeth cleaning, the media guidance application may search for a media asset with a play length that matches the determined length of time.

The media guidance application may then determine a current amount of time remaining in the teeth cleaning based on a current condition. For example, the user device may periodically determine the total surface area of the teeth that still needs to be cleaned or determine an amount of dental plaque that still needs to be removed and calculate a new amount of time remaining in the teeth cleaning based the latest determination.

The media guidance application may compare the current amount of time to the remaining play length. For example, the media guidance application may determine whether the initial amount of time corresponds to the current amount of time in order to determine whether or not the user is on schedule.

The media guidance application may then adjust the play length of the media asset to rectify the discrepancy. For example, in response to determining that the teeth cleaning will take longer (or shorter) than initially expected, the media guidance application may modify the length of time it takes for the user to consume a media asset that accompanies the activity. For example, the media guidance application may extend the play length of the media asset by including additional content. In another example, the media guidance application may extend the play length of the media asset by reducing a playback rate associated with the media asset. In another example, the media guidance application may decrease the play length of the media asset by advancing a current point of playback in the media asset to a later point of playback in the media asset. In yet another example, the media guidance application may decrease the play length of the media asset by cropping a portion of media asset.

The media guidance application may also perform other functions. One of the functions of the media guidance application is to provide media guidance data to users. As referred to herein, the phrase “media guidance data” or “guidance data” should be understood to mean any data related to content or data used in operating the guidance application. For example, the guidance data may include program information, guidance application settings, user preferences, user profile information, media listings, media-related information (e.g., broadcast times, broadcast channels, titles, descriptions, ratings information (e.g., parental control ratings, critic's ratings, etc.), genre or category information, actor information, logo data for broadcasters' or providers' logos, etc.), media format (e.g., standard definition, high definition, 3D, etc.), advertisement information (e.g., text, images, media clips, etc.), on-demand information, blogs, websites, and any other type of guidance data that is helpful for a user to navigate among and locate desired content selections.

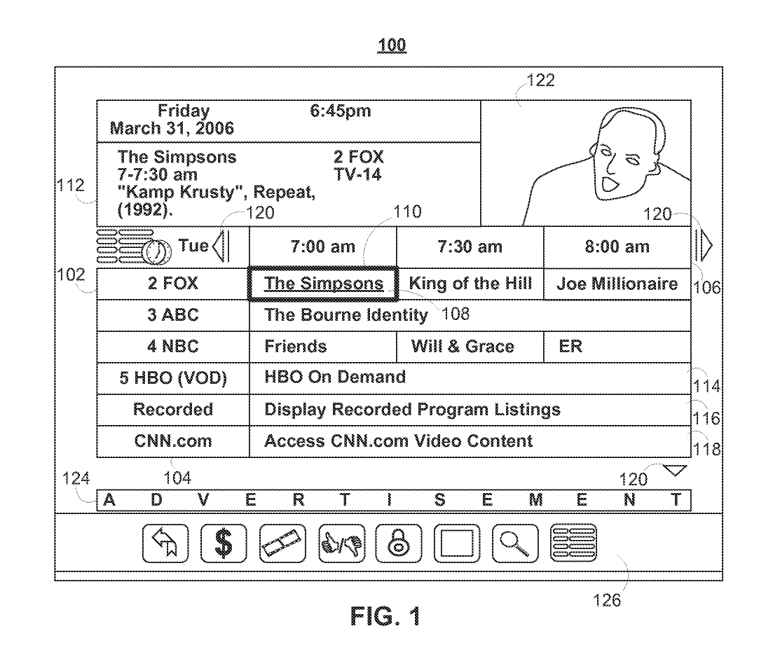

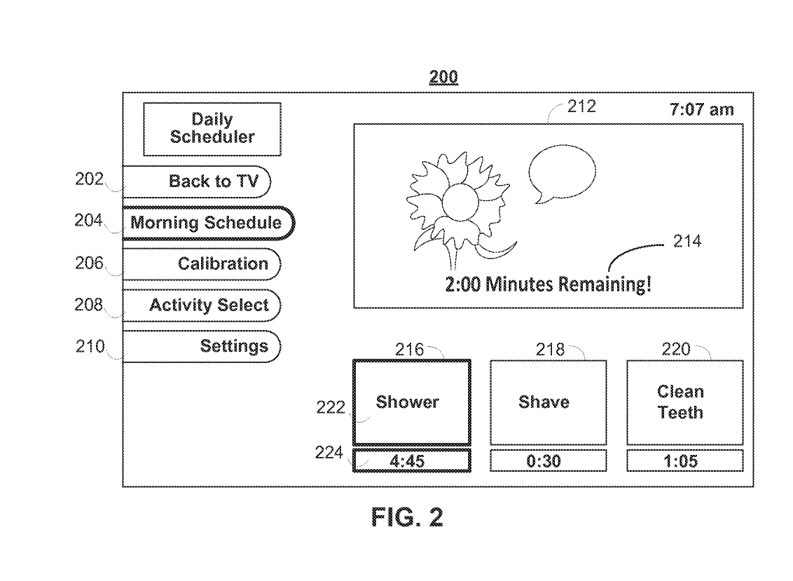

FIGS. 1-2 show illustrative display screens that may be used to provide media guidance data. The display screens shown in FIGS. 1-2 may be implemented on any suitable user equipment device or platform. While the displays of FIGS. 1-2 are illustrated as full screen displays, they may also be fully or partially overlaid over content being displayed. A user may indicate a desire to access content information by selecting a selectable option provided in a display screen (e.g., a menu option, a listings option, an icon, a hyperlink, etc.) or pressing a dedicated button (e.g., a GUIDE button) on a remote control or other user input interface or device. In response to the user's indication, the media guidance application may provide a display screen with media guidance data organized in one of several ways, such as by time and channel in a grid, by time, by channel, by source, by content type, by category (e.g., movies, sports, news, children, or other categories of programming), or other predefined, user-defined, or other organization criteria.

FIG. 1 shows illustrative grid program listings display 100 arranged by time and channel that also enables access to different types of content in a single display. Display 100 may include grid 102 with: (1) a column of channel/content type identifiers 104, where each channel/content type identifier (which is a cell in the column) identifies a different channel or content type available; and (2) a row of time identifiers 106, where each time identifier (which is a cell in the row) identifies a time block of programming. Grid 102 also includes cells of program listings, such as program listing 108, where each listing provides the title of the program provided on the listing's associated channel and time. With a user input device, a user can select program listings by moving highlight region 110. Information relating to the program listing selected by highlight region 110 may be provided in program information region 112. Region 112 may include, for example, the program title, the program description, the time the program is provided (if applicable), the channel the program is on (if applicable), the program's rating, and other desired information.

In addition to providing access to linear programming (e.g., content that is scheduled to be transmitted to a plurality of user equipment devices at a predetermined time and is provided according to a schedule), the media guidance application also provides access to non-linear programming (e.g., content accessible to a user equipment device at any time and is not provided according to a schedule). Non-linear programming may include content from different content sources including on-demand content (e.g., VOD), Internet content (e.g., streaming media, downloadable media, etc.), locally stored content (e.g., content stored on any user equipment device described above or other storage device), or other time-independent content. On-demand content may include movies or any other content provided by a particular content provider (e.g., HBO On Demand providing “The Sopranos” and “Curb Your Enthusiasm”). HBO ON DEMAND is a service mark owned by Time Warner Company L.P. et al. and THE SOPRANOS and CURB YOUR ENTHUSIASM are trademarks owned by the Home Box Office, Inc. Internet content may include web events, such as a chat session or Webcast, or content available on-demand as streaming content or downloadable content through an Internet web site or other Internet access (e.g. FTP).

Grid 102 may provide media guidance data for non-linear programming including on-demand listing 114, recorded content listing 116, and Internet content listing 118. A display combining media guidance data for content from different types of content sources is sometimes referred to as a “mixed-media” display. Various permutations of the types of media guidance data that may be displayed that are different than display 100 may be based on user selection or guidance application definition (e.g., a display of only recorded and broadcast listings, only on-demand and broadcast listings, etc.). As illustrated, listings 114, 116, and 118 are shown as spanning the entire time block displayed in grid 102 to indicate that selection of these listings may provide access to a display dedicated to on-demand listings, recorded listings, or Internet listings, respectively. In some embodiments, listings for these content types may be included directly in grid 102. Additional media guidance data may be displayed in response to the user selecting one of the navigational icons 120. (Pressing an arrow key on a user input device may affect the display in a similar manner as selecting navigational icons 120.)

Display 100 may also include video region 122, advertisement 124, and options region 126. Video region 122 may allow the user to view and/or preview programs that are currently available, will be available, or were available to the user. The content of video region 122 may correspond to, or be independent from, one of the listings displayed in grid 102. Grid displays including a video region are sometimes referred to as picture-in-guide (PIG) displays. PIG displays and their functionalities are described in greater detail in Satterfield et al. U.S. Pat. No. 6,564,378, issued May 13, 2003 and Yuen et al. U.S. Pat. No. 6,239,794, issued May 29, 2001, which are hereby incorporated by reference herein in their entireties. PIG displays may be included in other media guidance application display screens of the embodiments described herein.

Advertisement 124 may provide an advertisement for content that, depending on a viewer's access rights (e.g., for subscription programming), is currently available for viewing, will be available for viewing in the future, or may never become available for viewing, and may correspond to or be unrelated to one or more of the content listings in grid 102. Advertisement 124 may also be for products or services related or unrelated to the content displayed in grid 102. Advertisement 124 may be selectable and provide further information about content, provide information about a product or a service, enable purchasing of content, a product, or a service, provide content relating to the advertisement, etc. Advertisement 124 may be targeted based on a user's profile/preferences, monitored user activity, the type of display provided, or on other suitable targeted advertisement bases.

While advertisement 124 is shown as rectangular or banner shaped, advertisements may be provided in any suitable size, shape, and location in a guidance application display. For example, advertisement 124 may be provided as a rectangular shape that is horizontally adjacent to grid 102. This is sometimes referred to as a panel advertisement. In addition, advertisements may be overlaid over content or a guidance application display or embedded within a display. Advertisements may also include text, images, rotating images, video clips, or other types of content described above. Advertisements may be stored in a user equipment device having a guidance application, in a database connected to the user equipment, in a remote location (including streaming media servers), or on other storage means, or a combination of these locations. Providing advertisements in a media guidance application is discussed in greater detail in, for example, Knudson et al., U.S. Patent Application Publication No. 2003/0110499, filed Jan. 17, 2003; Ward, III et al. U.S. Pat. No. 6,756,997, issued Jun. 29, 2004; and Schein et al. U.S. Pat. No. 6,388,714, issued May 14, 2002, which are hereby incorporated by reference herein in their entireties. It will be appreciated that advertisements may be included in other media guidance application display screens of the embodiments described herein.

Options region 126 may allow the user to access different types of content, media guidance application displays, and/or media guidance application features. Options region 126 may be part of display 100 (and other display screens described herein), or may be invoked by a user by selecting an on-screen option or pressing a dedicated or assignable button on a user input device. The selectable options within options region 126 may concern features related to program listings in grid 102 or may include options available from a main menu display. Features related to program listings may include searching for other air times or ways of receiving a program, recording a program, enabling series recording of a program, setting program and/or channel as a favorite, purchasing a program, or other features. Options available from a main menu display may include search options, VOD options, parental control options, Internet options, cloud-based options, device synchronization options, second screen device options, options to access various types of media guidance data displays, options to subscribe to a premium service, options to edit a user's profile, options to access a browse overlay, or other options.

The media guidance application may be personalized based on a user's preferences. A personalized media guidance application allows a user to customize displays and features to create a personalized “experience” with the media guidance application. This personalized experience may be created by allowing a user to input these customizations and/or by the media guidance application monitoring user activity to determine various user preferences. Users may access their personalized guidance application by logging in or otherwise identifying themselves to the guidance application. Customization of the media guidance application may be made in accordance with a user profile. The customizations may include varying presentation schemes (e.g., color scheme of displays, font size of text, etc.), aspects of content listings displayed (e.g., only HDTV or only 3D programming, user-specified broadcast channels based on favorite channel selections, re-ordering the display of channels, recommended content, etc.), desired recording features (e.g., recording or series recordings for particular users, recording quality, etc.), parental control settings, customized presentation of Internet content (e.g., presentation of social media content, e-mail, electronically delivered articles, etc.) and other desired customizations.

The media guidance application may allow a user to provide user profile information or may automatically compile user profile information. The media guidance application may, for example, monitor the content the user accesses and/or other interactions the user may have with the guidance application. Additionally, the media guidance application may obtain all or part of other user profiles that are related to a particular user (e.g., from other web sites on the Internet the user accesses, such as www.allrovi.com, from other media guidance applications the user accesses, from other interactive applications the user accesses, from another user equipment device of the user, etc.), and/or obtain information about the user from other sources that the media guidance application may access. As a result, a user can be provided with a unified guidance application experience across the user's different user equipment devices. This type of user experience is described in greater detail below in connection with FIG. 4. Additional personalized media guidance application features are described in greater detail in Ellis et al., U.S. Patent Application Publication No. 2005/0251827, filed Jul. 11, 2005, Boyer et al., U.S. Pat. No. 7,165,098, issued Jan. 16, 2007, and Ellis et al., U.S. Patent Application Publication No. 2002/0174430, filed Feb. 21, 2002, which are hereby incorporated by reference herein in their entireties.

Another display arrangement for providing media guidance is shown in FIG. 2. Video mosaic display 200 includes selectable options 202 through 210. In display 200, option 204 is selected. In response to the selection of option 204 labeled “Morning Schedule,” media asset 212 is currently presented. For example, as indicated by the highlight of activity 216, the user is currently performing activity 216.

Display 200 includes two cells for activity 216. For example, cell 222 indicates an activity to be performed and cell 224 indicates a time associated with the activity. For example, the media guidance application has budgeted four minutes and forty-five seconds for the current activity, “Shower.” Furthermore, the media guidance application may present media asset 212, which corresponds to the current activity.

The media guidance application has also generated for display user progress feedback 214. For example, user progress feedback 214 indicates to a user that the user has two minutes remaining for this activity. The media guidance application also indicates other activities (e.g., represented by activity 218 and 220) that the user is scheduled to perform.

In some embodiments, the media guidance application may provide graphical images including cover art, still images from the content, video clip previews, live video from the content, or other types of content that indicate to a user the content that is to be presented during an activity. Each of the graphical listings may also be accompanied by text to provide further information about the content associated with the activity. Various systems and methods for graphically accentuating content are discussed in, for example, Yates, U.S. Patent Application Publication No. 2010/0153885, filed Dec. 29, 2005, which is hereby incorporated by reference herein in its entirety.

Display 200 also includes option 202. In response to selecting option 202, the media guidance application may return to a full-screen display of media asset 212 or otherwise exit out of the “Daily Scheduler.” In some embodiments, display 200 may automatically generate for display 200 in response to determining that a user has begun an activity and/or a user is scheduled to begin an activity. For example, the media guidance application may generate one or more reminders/alerts to indicate to a user that the user should be performing an activity that the user is currently not performing.

Display 200 also includes option 206. In response to selecting option 206, the media guidance application may calibrate itself to the actions of the user or receive input from the user regarding the user's actions. For example, a detection module incorporated into, or accessible by, the media guidance application may calibrate itself to the actions and movements of the user to better determine when a user is performing an activity and/or what activity the user is performing.

Display 200 also includes option 208. In response to selecting option 208, the media guidance application may allow the user to select an activity that the user is performing or wishes to perform. For example, the media guidance application may allow a user to “check in” or otherwise schedule an activity. Based on the input, the media guidance application may search for an appropriate media asset. The media guidance application may also receive information regarding how long a particular activity should take.

Display 200 also includes option 210. In response to selecting option 210, the media guidance application may allow the user to enter settings relating to monitoring the user. For example, the media guidance application may allow a user to turn on or turn off the monitoring of the user during certain times. The media guidance application may also receive user inputs for particular criteria that should be used when selecting one or more media assets. For example, the user may indicate a particular output type of media assets that the user enjoys when performing a particular activity. In another example, the media guidance application may receive a user input indicating a user preference for how media assets should be adjusted.

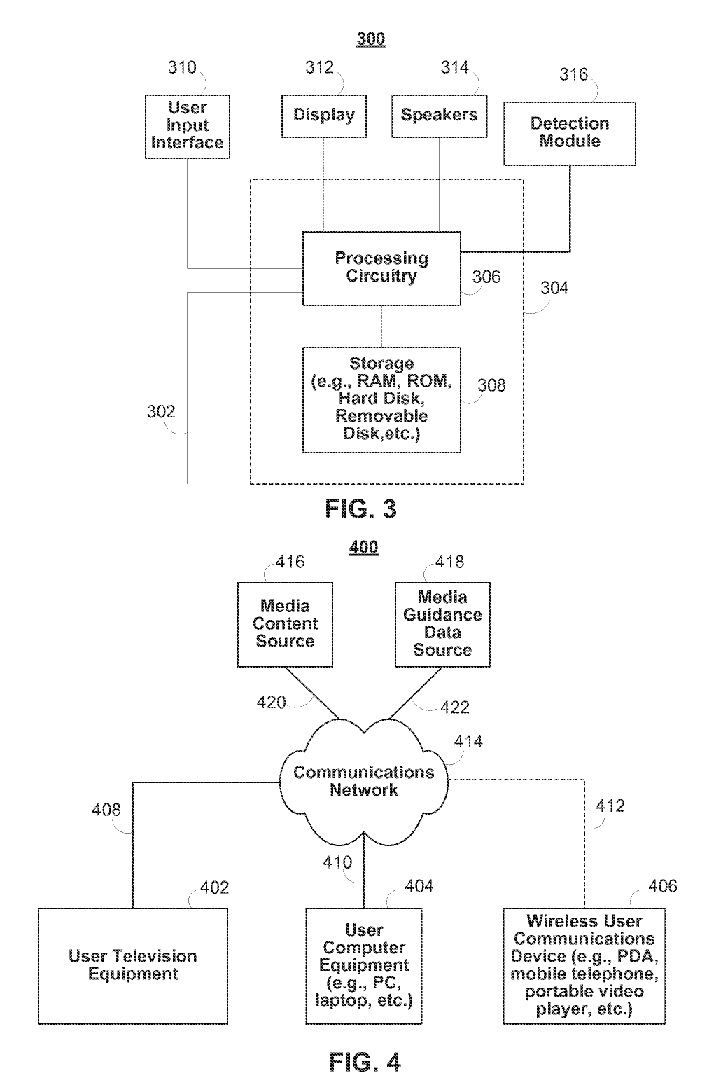

Users may access content and the media guidance application (and its display screens described above and below) from one or more of their user equipment devices. FIG. 3 shows a generalized embodiment of illustrative user equipment device 300. More specific implementations of user equipment devices are discussed below in connection with FIG. 4. User equipment device 300 may receive content and data via input/output (hereinafter “I/O”) path 302. I/O path 302 may provide content (e.g., broadcast programming, on-demand programming, Internet content, content available over a local area network (LAN) or wide area network (WAN), and/or other content) and data to control circuitry 304, which includes processing circuitry 306 and storage 308. Control circuitry 304 may be used to send and receive commands, requests, and other suitable data using I/O path 302. I/O path 302 may connect control circuitry 304 (and specifically processing circuitry 306) to one or more communications paths (described below). I/O functions may be provided by one or more of these communications paths, but are shown as a single path in FIG. 3 to avoid overcomplicating the drawing.

Control circuitry 304 may be based on any suitable processing circuitry such as processing circuitry 306. As referred to herein, processing circuitry should be understood to mean circuitry based on one or more microprocessors, microcontrollers, digital signal processors, programmable logic devices, field-programmable gate arrays (FPGAs), application-specific integrated circuits (ASICs), etc., and may include a multi-core processor (e.g., dual-core, quad-core, hexa-core, or any suitable number of cores) or supercomputer. In some embodiments, processing circuitry may be distributed across multiple separate processors or processing units, for example, multiple of the same type of processing units (e.g., two Intel Core i7 processors) or multiple different processors (e.g., an Intel Core i5 processor and an Intel Core i7 processor). In some embodiments, control circuitry 304 executes instructions for a media guidance application stored in memory (i.e., storage 308). Specifically, control circuitry 304 may be instructed by the media guidance application to perform the functions discussed above and below. For example, the media guidance application may provide instructions to control circuitry 304 to generate the media guidance displays. In some implementations, any action performed by control circuitry 304 may be based on instructions received from the media guidance application.

In client-server based embodiments, control circuitry 304 may include communications circuitry suitable for communicating with a guidance application server or other networks or servers. The instructions for carrying out the above mentioned functionality may be stored on the guidance application server. Communications circuitry may include a cable modem, an integrated services digital network (ISDN) modem, a digital subscriber line (DSL) modem, a telephone modem, Ethernet card, or a wireless modem for communications with other equipment, or any other suitable communications circuitry. Such communications may involve the Internet or any other suitable communications networks or paths (which is described in more detail in connection with FIG. 4). In addition, communications circuitry may include circuitry that enables peer-to-peer communication of user equipment devices, or communication of user equipment devices in locations remote from each other (described in more detail below).

Memory may be an electronic storage device provided as storage 308 that is part of control circuitry 304. As referred to herein, the phrase “electronic storage device” or “storage device” should be understood to mean any device for storing electronic data, computer software, or firmware, such as random-access memory, read-only memory, hard drives, optical drives, digital video disc (DVD) recorders, compact disc (CD) recorders, BLU-RAY disc (BD) recorders, BLU-RAY 3D disc recorders, digital video recorders (DVR, sometimes called a personal video recorder, or PVR), solid state devices, quantum storage devices, gaming consoles, gaming media, or any other suitable fixed or removable storage devices, and/or any combination of the same. Storage 308 may be used to store various types of content described herein as well as media guidance data described above. Nonvolatile memory may also be used (e.g., to launch a boot-up routine and other instructions). Cloud-based storage, described in relation to FIG. 4, may be used to supplement storage 308 or instead of storage 308.

Control circuitry 304 may include video generating circuitry and tuning circuitry, such as one or more analog tuners, one or more MPEG-2 decoders or other digital decoding circuitry, high-definition tuners, or any other suitable tuning or video circuits or combinations of such circuits. Encoding circuitry (e.g., for converting over-the-air, analog, or digital signals to MPEG signals for storage) may also be provided. Control circuitry 304 may also include scaler circuitry for upconverting and downconverting content into the preferred output format of the user equipment 300. Circuitry 304 may also include digital-to-analog converter circuitry and analog-to-digital converter circuitry for converting between digital and analog signals. The tuning and encoding circuitry may be used by the user equipment device to receive and to display, to play, or to record content. The tuning and encoding circuitry may also be used to receive guidance data. The circuitry described herein, including for example, the tuning, video generating, encoding, decoding, encrypting, decrypting, scaler, and analog/digital circuitry, may be implemented using software running on one or more general purpose or specialized processors. Multiple tuners may be provided to handle simultaneous tuning functions (e.g., watch and record functions, picture-in-picture (PIP) functions, multiple-tuner recording, etc.). If storage 308 is provided as a separate device from user equipment 300, the tuning and encoding circuitry (including multiple tuners) may be associated with storage 308.

A user may send instructions to control circuitry 304 using user input interface 310. User input interface 310 may be any suitable user interface, such as a remote control, mouse, trackball, keypad, keyboard, touch screen, touchpad, stylus input, joystick, voice recognition interface, or other user input interfaces. Display 312 may be provided as a stand-alone device or integrated with other elements of user equipment device 300. For example, display 312 may be a touchscreen or touch-sensitive display. In such circumstances, user input interface 312 may be integrated with or combined with display 312. Display 312 may be one or more of a monitor, a television, a liquid crystal display (LCD) for a mobile device, amorphous silicon display, low temperature poly silicon display, electronic ink display, electrophoretic display, active matrix display, electro-wetting display, electrofluidic display, cathode ray tube display, light-emitting diode display, electroluminescent display, plasma display panel, high-performance addressing display, thin-film transistor display, organic light-emitting diode display, surface-conduction electron-emitter display (SED), laser television, carbon nanotubes, quantum dot display, interferometric modulator display, or any other suitable equipment for displaying visual images. In some embodiments, display 312 may be HDTV-capable. In some embodiments, display 312 may be a 3D display, and the interactive media guidance application and any suitable content may be displayed in 3D. A video card or graphics card may generate the output to the display 312. The video card may offer various functions such as accelerated rendering of 3D scenes and 2D graphics, MPEG-2/MPEG-4 decoding, TV output, or the ability to connect multiple monitors. The video card may be any processing circuitry described above in relation to control circuitry 304. The video card may be integrated with the control circuitry 304. Speakers 314 may be provided as integrated with other elements of user equipment device 300 or may be stand-alone units. The audio component of videos and other content displayed on display 312 may be played through speakers 314. In some embodiments, the audio may be distributed to a receiver (not shown), which processes and outputs the audio via speakers 314.

User device also includes detection module 316 for use in determining whether or not a user is performing an activity, the progress of the user in the activity, and/or a condition associated with the activity. In some embodiments, detection module 316 may also monitor physical movement of the user and/or sounds. Detection module 316 may use any suitable sensor to detect movement, including, but not limited to, an optical sensor or an infrared sensor. The movement sensor may detect movement at a specific frequency or framerate, and the frequency or framerate may be either fixed or variable. For example, the movement sensor may detect movement at a first framerate, and in response to detecting movement of a user (e.g., a hand motion, a user entering an area, a user holding an object, etc.), the movement sensor may increase the framerate to a second framerate that is higher than the first framerate. The movement sensor may continue to detect movement at the second, increased framerate for a set period of time before reverting to the first framerate. The movement sensor may revert back to the first framerate after a period of time of detecting no movement. In some embodiments, an audio sensor may be used to detect sound from the user or the user's environment. The audio sensor may detect volume, frequency, pitch, tone, or any other audio characteristics. The media guidance application may further be configured with speech recognition software to enable the media guidance application to recognize any words or phrases spoken by the user for use in determining whether or not a user is performing an activity, the progress of the user in the activity, and/or an condition associated with the activity. As an illustrative example, an increased volume for a short amount of time emanating from the user (e.g., a cheer) in conjunction with increased physical movement (e.g., a first pump) may indicate a user's interest in a particular portion of the media asset that the user is currently watching.

Detection module 316 may further include various components (e.g., a video detection component, an audio detection component, etc.) for determining and/or detecting other types of information. In some embodiments, detection module 316 may include an audio/video detection component, which determines or receives information describing objects in images and/or noise emanating from a user or media asset.

For example, detection module 316 may include one or more content-recognition modules, which may be used by the media guidance application to analyze information received from a content capture device (e.g., video and/or audio recorder). For example, the media guidance application may include an object recognition module. The object recognition module may use edge detection, pattern recognition, including, but not limited to, self-learning systems (e.g., neural networks), optical character recognition, on-line character recognition (including, but not limited to, dynamic character recognition, real-time character recognition, intelligent character recognition), and/or any other suitable technique or method to determine the objects in and/or characteristics of video and audio recordings. For example, the media guidance application may receive a media asset in the form of a video of the actions of a user. The video may include a series of frames. For each frame of the video, the media guidance application may use an object recognition module to determine the characteristics associated with each frame (or the media assets as a whole) of the video to determine whether or not a user performing an activity, the progress of the user in the activity, and/or a condition associated with the activity.

In some embodiments, the content-recognition module or algorithm may also include audio analysis and speech recognition techniques, including, but not limited to, Hidden Markov Models, dynamic time warping, and/or neural networks (as described above) to process audio data and/or translate spoken words into text. The content-recognition module may also use any other suitable techniques for processing audio and/or visual data. For example, the content-recognition module may analyze audio data to determine whether or not a user is talking. Furthermore, the content-recognition module may analyze video and/or audio data to determine whether or not a user is performing an activity, the progress of the user in the activity, and/or a condition associated with the activity.

In addition, the media guidance application may use multiple types of optical character recognition and/or fuzzy logic, for example, when comparing multiple data fields (e.g., as contained in databases described below). For example, after the content-recognition module or algorithm translates video and/or audio recordings into text, the media guidance application (e.g., via control circuitry 304) may cross-reference the translated text with a database (e.g., located at storage 308 or media guidance data source 418 (FIG. 4)) to determine whether or not the translated text corresponds to a user performing an activity, the progress of the user in the activity, and/or a condition associated with the activity.

For example, the media guidance application may arrange the text into data fields and cross-reference the data fields with other data fields (e.g., in a lookup table database) corresponding to possible values associated with a keyword in a media asset, the system may determine two fields and/or values to be identical even though the substance of the data field or value (e.g., two different spellings) is not identical. In some embodiments, the system may analyze particular data fields of a data structure or media asset frame for particular values or text. The data fields could be associated with characteristics, other data, and/or any other information required for the function of the embodiments described herein. Furthermore, the data fields could contain values (e.g., the data fields could be expressed in binary or any other suitable code or programming language).

In some embodiments, detection module 316 may include a Global Positioning System (“GPS”) detection component, which determines or receives information describing the geographic position of a user. For example, the GPS detection component may, additionally or alternatively, determine whether or not the user is performing an activity (e.g., sleeping) based on the determined location (e.g., a user's bed) of the user.

In some embodiments, detection module 316 may include an Internet access component, which determines whether or not a user is performing an activity, the progress of the user in the activity, and/or a condition associated with the activity. For example, the Internet access component may, additionally or alternatively, determine whether or not information on the Internet (e.g., social media updates posted by the user) indicates a current activity of a user. For example, the user may post that he or she is currently performing an activity or may “check in” to a particular activity.

In some embodiments, the media guidance application may receive information from a particular source (e.g., a website, a profile associated with a user, a content provider, a social media network, etc.) for use in determining whether or not a user is performing an activity, the progress of the user in the activity, and/or a condition associated with the activity. For example, the media guidance application may retrieve a list of friends (e.g., a social network buddy list), contacts (e.g., retrieved from a phone/text message/e-mail account associated with the user), and/or other listings featuring other entities with known associations to the user and obtain information used to determine whether or not a user is performing an activity, the progress of the user in the activity, and/or a condition associated with the activity based on this information.

As used herein, a “social network” refers to a platform that facilitates networking and/or social relations among people who, for example, share interests, activities, backgrounds, and/or real-life connections. In some cases, social networks may facilitate communication between multiple user devices (e.g., computers, televisions, smartphones, tablets, etc.) associated with different users by exchanging content from one device to another via a social media server. As used herein, a “social media server” refers to a computer server that facilitates a social network. For example, a social media server owned/operated/used by a social media provider may make content (e.g., status updates, microblog posts, images, graphic messages, etc.) associated with a first user accessible to a second user that is within the same social network as the first user.

In some embodiments, detection module 316 may include a device access component, which receives information from other devices (e.g., an electronic toothbrush) to determine whether or not a user is performing an activity, the progress of the user in the activity, and/or a condition associated with the activity. For example, the device access component may receive information received from another device that indicates that a user is using that particular device. For example, if the media guidance application receives information from a television indicating that the user recently turned the television on or a user is currently viewing/interacting with the television, the media guidance application may determine that the user is not currently performing another activity (e.g., attending school). In another example, the media guidance application may access sensors in other user devices. For example, the media guidance application may access motion-sensors in a home alarm system to determine whether the user is at home. In another example, the media guidance application may access sensors that monitor the amount of dental plaque in the mouth of a user, the amount of surface area of teeth remaining to be cleaned, the number of germs on the hands of a user, the amount of dust on furniture, etc.

The guidance application may be implemented using any suitable architecture. For example, it may be a stand-alone application wholly implemented on user equipment device 300. In such an approach, instructions of the application are stored locally (e.g., in storage 308), and data for use by the application is downloaded on a periodic basis (e.g., from an out-of-band feed, from an Internet resource, or using another suitable approach). Control circuitry 304 may retrieve instructions of the application from storage 308 and process the instructions to generate any of the displays discussed herein. Based on the processed instructions, control circuitry 304 may determine what action to perform when input is received from input interface 310. For example, movement of a cursor on a display up/down may be indicated by the processed instructions when input interface 310 indicates that an up/down button was selected.

In some embodiments, the media guidance application is a client-server based application. Data for use by a thick or thin client implemented on user equipment device 300 is retrieved on-demand by issuing requests to a server remote to the user equipment device 300. In one example of a client-server based guidance application, control circuitry 304 runs a web browser that interprets web pages provided by a remote server. For example, the remote server may store the instructions for the application in a storage device. The remote server may process the stored instructions using circuitry (e.g., control circuitry 304) and generate the displays discussed above and below. The client device may receive the displays generated by the remote server and may display the content of the displays locally on equipment device 300. This way, the processing of the instructions is performed remotely by the server while the resulting displays are provided locally on equipment device 300. Equipment device 300 may receive inputs from the user via input interface 310 and transmit those inputs to the remote server for processing and generating the corresponding displays. For example, equipment device 300 may transmit a communication to the remote server indicating that an up/down button was selected via input interface 310. The remote server may process instructions in accordance with that input and generate a display of the application corresponding to the input (e.g., a display that moves a cursor up/down). The generated display is then transmitted to equipment device 300 for presentation to the user.

In some embodiments, the media guidance application is downloaded and interpreted or otherwise run by an interpreter or virtual machine (run by control circuitry 304). In some embodiments, the guidance application may be encoded in the ETV Binary Interchange Format (EBIF), received by control circuitry 304 as part of a suitable feed, and interpreted by a user agent running on control circuitry 304. For example, the guidance application may be an EBIF application. In some embodiments, the guidance application may be defined by a series of JAVA-based files that are received and run by a local virtual machine or other suitable middleware executed by control circuitry 304. In some of such embodiments (e.g., those employing MPEG-2 or other digital media encoding schemes), the guidance application may be, for example, encoded and transmitted in an MPEG-2 object carousel with the MPEG audio and video packets of a program.

User equipment device 300 of FIG. 3 can be implemented in system 400 of FIG. 4 as user television equipment 402, user computer equipment 404, wireless user communications device 406, or any other type of user equipment suitable for accessing content, such as a non-portable gaming machine. For simplicity, these devices may be referred to herein collectively as user equipment or user equipment devices, and may be substantially similar to user equipment devices described above. User equipment devices, on which a media guidance application may be implemented, may function as a standalone device or may be part of a network of devices. Various network configurations of devices may be implemented and are discussed in more detail below.

A user equipment device utilizing at least some of the system features described above in connection with FIG. 3 may not be classified solely as user television equipment 402, user computer equipment 404, or a wireless user communications device 406. For example, user television equipment 402 may, like some user computer equipment 404, be Internet-enabled allowing for access to Internet content, while user computer equipment 404 may, like some television equipment 402, include a tuner allowing for access to television programming. The media guidance application may have the same layout on various different types of user equipment or may be tailored to the display capabilities of the user equipment. For example, on user computer equipment 404, the guidance application may be provided as a web site accessed by a web browser. In another example, the guidance application may be scaled down for wireless user communications devices 406.

In system 400, there is typically more than one of each type of user equipment device but only one of each is shown in FIG. 4 to avoid overcomplicating the drawing. In addition, each user may utilize more than one type of user equipment device and also more than one of each type of user equipment device.

In some embodiments, a user equipment device (e.g., user television equipment 402, user computer equipment 404, wireless user communications device 406) may be referred to as a “second screen device.” For example, a second screen device may supplement content presented on a first user equipment device. The content presented on the second screen device may be any suitable content that supplements the content presented on the first device. In some embodiments, the second screen device provides an interface for adjusting settings and display preferences of the first device. In some embodiments, the second screen device is configured for interacting with other second screen devices or for interacting with a social network. The second screen device can be located in the same room as the first device, a different room from the first device but in the same house or building, or in a different building from the first device.

The user may also set various settings to maintain consistent media guidance application settings across in-home devices and remote devices. Settings include those described herein, as well as channel and program favorites, programming preferences that the guidance application utilizes to make programming recommendations, display preferences, and other desirable guidance settings. For example, if a user sets a channel as a favorite on, for example, the web site www.allrovi.com on their personal computer at their office, the same channel would appear as a favorite on the user's in-home devices (e.g., user television equipment and user computer equipment) as well as the user's mobile devices, if desired. Therefore, changes made on one user equipment device can change the guidance experience on another user equipment device, regardless of whether they are the same or a different type of user equipment device. In addition, the changes made may be based on settings input by a user, as well as user activity monitored by the guidance application.

The user equipment devices may be coupled to communications network 414. Namely, user television equipment 402, user computer equipment 404, and wireless user communications device 406 are coupled to communications network 414 via communications paths 408, 410, and 412, respectively. Communications network 414 may be one or more networks including the Internet, a mobile phone network, mobile voice or data network (e.g., a 4G or LTE network), cable network, public switched telephone network, or other types of communications network or combinations of communications networks. Paths 408, 410, and 412 may separately or together include one or more communications paths, such as, a satellite path, a fiber-optic path, a cable path, a path that supports Internet communications (e.g., IPTV), free-space connections (e.g., for broadcast or other wireless signals), or any other suitable wired or wireless communications path or combination of such paths. Path 412 is drawn with dotted lines to indicate that in the exemplary embodiment shown in FIG. 4 it is a wireless path and paths 408 and 410 are drawn as solid lines to indicate they are wired paths (although these paths may be wireless paths, if desired). Communications with the user equipment devices may be provided by one or more of these communications paths, but are shown as a single path in FIG. 4 to avoid overcomplicating the drawing.

Although communications paths are not drawn between user equipment devices, these devices may communicate directly with each other via communication paths, such as those described above in connection with paths 408, 410, and 412, as well as other short-range point-to-point communication paths, such as USB cables, IEEE 1394 cables, wireless paths (e.g., Bluetooth, infrared, IEEE 802-11x, etc.), or other short-range communication via wired or wireless paths. BLUETOOTH is a certification mark owned by Bluetooth SIG, INC. The user equipment devices may also communicate with each other directly through an indirect path via communications network 414.

System 400 includes content source 416 and media guidance data source 418 coupled to communications network 414 via communication paths 420 and 422, respectively. Paths 420 and 422 may include any of the communication paths described above in connection with paths 408, 410, and 412. Communications with the content source 416 and media guidance data source 418 may be exchanged over one or more communications paths, but are shown as a single path in FIG. 4 to avoid overcomplicating the drawing. In addition, there may be more than one of each of content source 416 and media guidance data source 418, but only one of each is shown in FIG. 4 to avoid overcomplicating the drawing. (The different types of each of these sources are discussed below.) If desired, content source 416 and media guidance data source 418 may be integrated as one source device. Although communications between sources 416 and 418 with user equipment devices 402, 404, and 406 are shown as through communications network 414, in some embodiments, sources 416 and 418 may communicate directly with user equipment devices 402, 404, and 406 via communication paths (not shown) such as those described above in connection with paths 408, 410, and 412.

Content source 416 may include one or more types of content distribution equipment including a television distribution facility, cable system headend, satellite distribution facility, programming sources (e.g., television broadcasters, such as NBC, ABC, HBO, etc.), intermediate distribution facilities and/or servers, Internet providers, on-demand media servers, and other content providers. NBC is a trademark owned by the National Broadcasting Company, Inc., ABC is a trademark owned by the American Broadcasting Company, Inc., and HBO is a trademark owned by the Home Box Office, Inc. Content source 416 may be the originator of content (e.g., a television broadcaster, a Webcast provider, etc.) or may not be the originator of content (e.g., an on-demand content provider, an Internet provider of content of broadcast programs for downloading, etc.). Content source 416 may include cable sources, satellite providers, on-demand providers, Internet providers, over-the-top content providers, or other providers of content. Content source 416 may also include a remote media server used to store different types of content (including video content selected by a user), in a location remote from any of the user equipment devices. Systems and methods for remote storage of content, and providing remotely stored content to user equipment are discussed in greater detail in connection with Ellis et al., U.S. Pat. No. 7,761,892, issued Jul. 20, 2010, which is hereby incorporated by reference herein in its entirety.

Media guidance data source 418 may provide media guidance data, such as the media guidance data described above. Media guidance data may be provided to the user equipment devices using any suitable approach. In some embodiments, the guidance application may be a stand-alone interactive television program guide that receives program guide data via a data feed (e.g., a continuous feed or trickle feed). Program schedule data and other guidance data may be provided to the user equipment on a television channel sideband, using an in-band digital signal, using an out-of-band digital signal, or by any other suitable data transmission technique. Program schedule data and other media guidance data may be provided to user equipment on multiple analog or digital television channels.

In some embodiments, guidance data from media guidance data source 418 may be provided to users' equipment using a client-server approach. For example, a user equipment device may pull media guidance data from a server, or a server may push media guidance data to a user equipment device. In some embodiments, a guidance application client residing on the user's equipment may initiate sessions with source 418 to obtain guidance data when needed, e.g., when the guidance data is out of date or when the user equipment device receives a request from the user to receive data. Media guidance may be provided to the user equipment with any suitable frequency (e.g., continuously, daily, a user-specified period of time, a system-specified period of time, in response to a request from user equipment, etc.). Media guidance data source 418 may provide user equipment devices 402, 404, and 406 the media guidance application itself or software updates for the media guidance application.

In some embodiments, the media guidance data may include viewer data. For example, the viewer data may include current and/or historical user activity information (e.g., what content the user typically watches, what times of day the user watches content, whether the user interacts with a social network, at what times the user interacts with a social network to post information, what types of content the user typically watches (e.g., pay TV or free TV), mood, brain activity information, etc.). The media guidance data may also include subscription data. For example, the subscription data may identify which sources or services a given user subscribes to and/or which sources or services the given user has previously subscribed to but later terminated access (e.g., whether the user subscribes to premium channels, whether the user has added a premium level of services, whether the user has increased Internet speed). In some embodiments, the viewer data and/or the subscription data may identify patterns of a given user for a period of more than one year. The media guidance data may include a model (e.g., a survivor model) used for generating a score that indicates a likelihood a given user will terminate access to a service/source. For example, the media guidance application may process the viewer data with the subscription data using the model to generate a value or score that indicates a likelihood of whether the given user will terminate access to a particular service or source. In particular, a higher score may indicate a higher level of confidence that the user will terminate access to a particular service or source. Based on the score, the media guidance application may generate promotions and advertisements that entice the user to keep the particular service or source indicated by the score as one to which the user will likely terminate access.

Media guidance applications may be, for example, stand-alone applications implemented on user equipment devices. For example, the media guidance application may be implemented as software or a set of executable instructions which may be stored in storage 308, and executed by control circuitry 304 of a user equipment device 300. In some embodiments, media guidance applications may be client-server applications where only a client application resides on the user equipment device, and server application resides on a remote server. For example, media guidance applications may be implemented partially as a client application on control circuitry 304 of user equipment device 300 and partially on a remote server as a server application (e.g., media guidance data source 418) running on control circuitry of the remote server. When executed by control circuitry of the remote server (such as media guidance data source 418), the media guidance application may instruct the control circuitry to generate the guidance application displays and transmit the generated displays to the user equipment devices. The server application may instruct the control circuitry of the media guidance data source 418 to transmit data for storage on the user equipment. The client application may instruct control circuitry of the receiving user equipment to generate the guidance application displays.

Content and/or media guidance data delivered to user equipment devices 402, 404, and 406 may be over-the-top (OTT) content. OTT content delivery allows Internet-enabled user devices, including any user equipment device described above, to receive content that is transferred over the Internet, including any content described above, in addition to content received over cable or satellite connections. OTT content is delivered via an Internet connection provided by an Internet service provider (ISP), but a third party distributes the content. The ISP may not be responsible for the viewing abilities, copyrights, or redistribution of the content, and may only transfer IP packets provided by the OTT content provider. Examples of OTT content providers include YOUTUBE, NETFLIX, and HULU, which provide audio and video via IP packets. Youtube is a trademark owned by Google Inc., Netflix is a trademark owned by Netflix Inc., and Hulu is a trademark owned by Hulu, LLC. OTT content providers may additionally or alternatively provide media guidance data described above. In addition to content and/or media guidance data, providers of OTT content can distribute media guidance applications (e.g., web-based applications or cloud-based applications), or the content can be displayed by media guidance applications stored on the user equipment device.

Media guidance system 400 is intended to illustrate a number of approaches, or network configurations, by which user equipment devices and sources of content and guidance data may communicate with each other for the purpose of accessing content and providing media guidance. The embodiments described herein may be applied in any one or a subset of these approaches, or in a system employing other approaches for delivering content and providing media guidance. The following four approaches provide specific illustrations of the generalized example of FIG. 4.

In one approach, user equipment devices may communicate with each other within a home network. User equipment devices can communicate with each other directly via short-range point-to-point communication schemes described above, via indirect paths through a hub or other similar device provided on a home network, or via communications network 414. Each of the multiple individuals in a single home may operate different user equipment devices on the home network. As a result, it may be desirable for various media guidance information or settings to be communicated between the different user equipment devices. For example, it may be desirable for users to maintain consistent media guidance application settings on different user equipment devices within a home network, as described in greater detail in Ellis et al., U.S. patent application Ser. No. 11/179,410, filed Jul. 11, 2005. Different types of user equipment devices in a home network may also communicate with each other to transmit content. For example, a user may transmit content from user computer equipment to a portable video player or portable music player.

In a second approach, users may have multiple types of user equipment by which they access content and obtain media guidance. For example, some users may have home networks that are accessed by in-home and mobile devices. Users may control in-home devices via a media guidance application implemented on a remote device. For example, users may access an online media guidance application on a website via a personal computer at their office, or a mobile device such as a PDA or web-enabled mobile telephone. The user may set various settings (e.g., recordings, reminders, or other settings) on the online guidance application to control the user's in-home equipment. The online guide may control the user's equipment directly, or by communicating with a media guidance application on the user's in-home equipment. Various systems and methods for user equipment devices communicating, where the user equipment devices are in locations remote from each other, is discussed in, for example, Ellis et al., U.S. Pat. No. 8,046,801, issued Oct. 25, 2011, which is hereby incorporated by reference herein in its entirety.

In a third approach, users of user equipment devices inside and outside a home can use their media guidance application to communicate directly with content source 416 to access content. Specifically, within a home, users of user television equipment 402 and user computer equipment 404 may access the media guidance application to navigate among and locate desirable content. Users may also access the media guidance application outside of the home using wireless user communications devices 406 to navigate among and locate desirable content.

In a fourth approach, user equipment devices may operate in a cloud computing environment to access cloud services. In a cloud computing environment, various types of computing services for content sharing, storage or distribution (e.g., video sharing sites or social networking sites) are provided by a collection of network-accessible computing and storage resources, referred to as “the cloud.” For example, the cloud can include a collection of server computing devices, which may be located centrally or at distributed locations, that provide cloud-based services to various types of users and devices connected via a network such as the Internet via communications network 414. These cloud resources may include one or more content sources 416 and one or more media guidance data sources 418. In addition or in the alternative, the remote computing sites may include other user equipment devices, such as user television equipment 402, user computer equipment 404, and wireless user communications device 406. For example, the other user equipment devices may provide access to a stored copy of a video or a streamed video. In such embodiments, user equipment devices may operate in a peer-to-peer manner without communicating with a central server.

The cloud provides access to services, such as content storage, content sharing, or social networking services, among other examples, as well as access to any content described above, for user equipment devices. Services can be provided in the cloud through cloud computing service providers, or through other providers of online services. For example, the cloud-based services can include a content storage service, a content sharing site, a social networking site, or other services via which user-sourced content is distributed for viewing by others on connected devices. These cloud-based services may allow a user equipment device to store content to the cloud and to receive content from the cloud rather than storing content locally and accessing locally-stored content.

A user may use various content capture devices, such as camcorders, digital cameras with video mode, audio recorders, mobile phones, and handheld computing devices, to record content. The user can upload content to a content storage service on the cloud either directly, for example, from user computer equipment 404 or wireless user communications device 406 having content capture feature. Alternatively, the user can first transfer the content to a user equipment device, such as user computer equipment 404. The user equipment device storing the content uploads the content to the cloud using a data transmission service on communications network 414. In some embodiments, the user equipment device itself is a cloud resource, and other user equipment devices can access the content directly from the user equipment device on which the user stored the content.

Cloud resources may be accessed by a user equipment device using, for example, a web browser, a media guidance application, a desktop application, a mobile application, and/or any combination of access applications of the same. The user equipment device may be a cloud client that relies on cloud computing for application delivery, or the user equipment device may have some functionality without access to cloud resources. For example, some applications running on the user equipment device may be cloud applications, i.e., applications delivered as a service over the Internet, while other applications may be stored and run on the user equipment device. In some embodiments, a user device may receive content from multiple cloud resources simultaneously. For example, a user device can stream audio from one cloud resource while downloading content from a second cloud resource. Or a user device can download content from multiple cloud resources for more efficient downloading. In some embodiments, user equipment devices can use cloud resources for processing operations such as the processing operations performed by processing circuitry described in relation to FIG. 3.

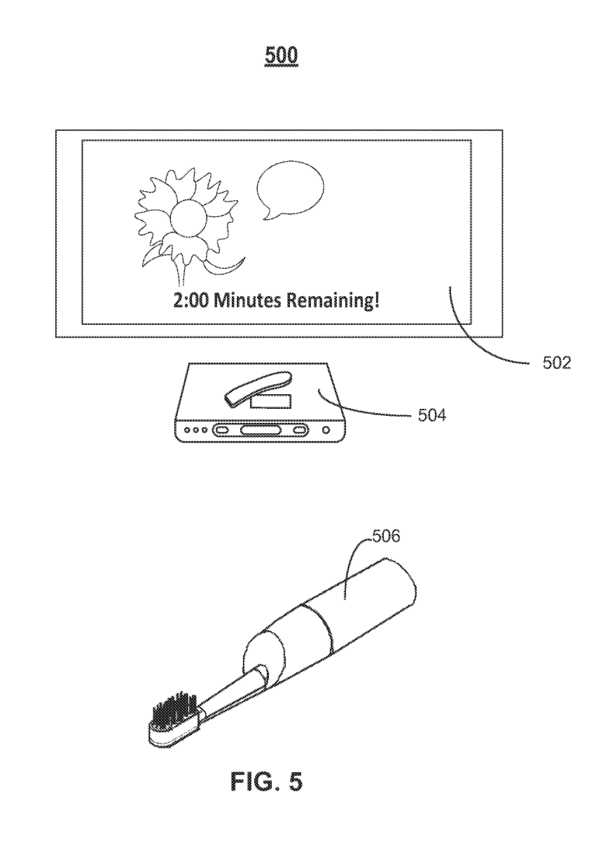

FIG. 5 is a illustration of a user device linked to the presentation of a media asset. For example, illustration 500 includes media asset 502, display device 504, and user device 506. For example, user device 506 may be an electric toothbrush, the use of which triggers the display of media asset 502 via display device 504. In some embodiments, display device 504 may correspond to user equipment devices 402, 404, and/or 406 (FIG. 4). Likewise, media asset 502 may correspond to media asset 212 (FIG. 2)).

User device 506 may incorporate one or more sensors (e.g., detection module 316 (FIG. 3)) for use in determining whether or not a user is performing an activity (e.g., using user device 506 to perform a teeth cleaning) and/or a current condition or progress of the user related to the activity. For example, the media guidance application (e.g., implemented on display device 504) may receive (e.g., via I/O path 302 (FIG. 3)) information from user device 506 indicating that the user has begun performing the activity. In response, the media guidance application may generate for display media asset 502. Alternatively, a media guidance application (e.g., implemented on user device 506) may detect that a user is currently using user device 506 and transmit a request to display device 504 to generate for display media asset 502.

In some embodiments, the media guidance application may continuously or periodically detect/receive information indicating the current progress of the user in the activity and/or one or more conditions related to the activity. For example, based on the detected/received information, the media guidance application may adjust the play length of media asset 502. Alternatively or additionally, in response to determining that a user has completed the activity, the media guidance application may generate for display a different media asset (e.g., corresponding to a different activity) than media asset 502.

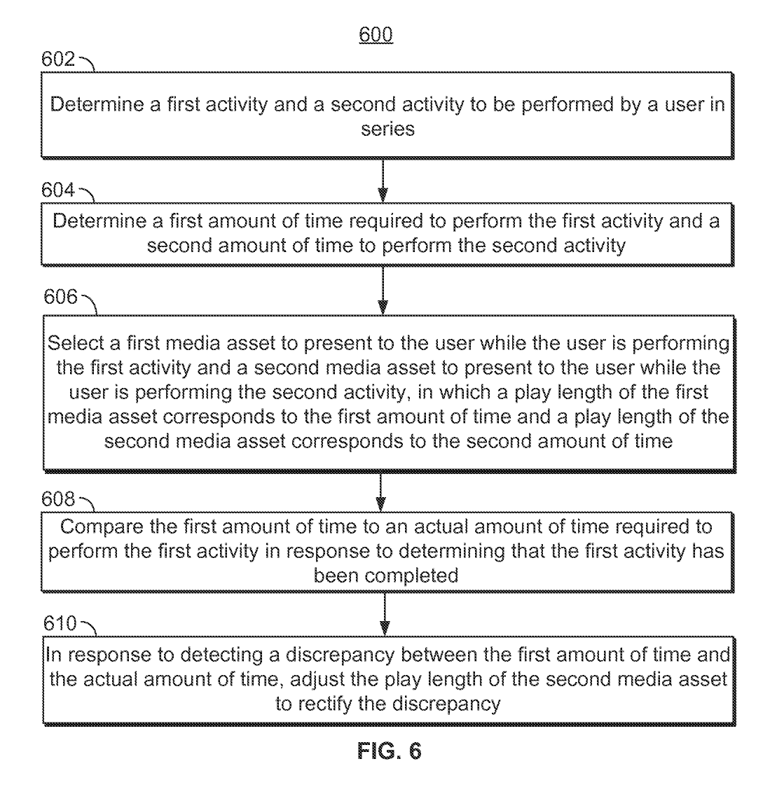

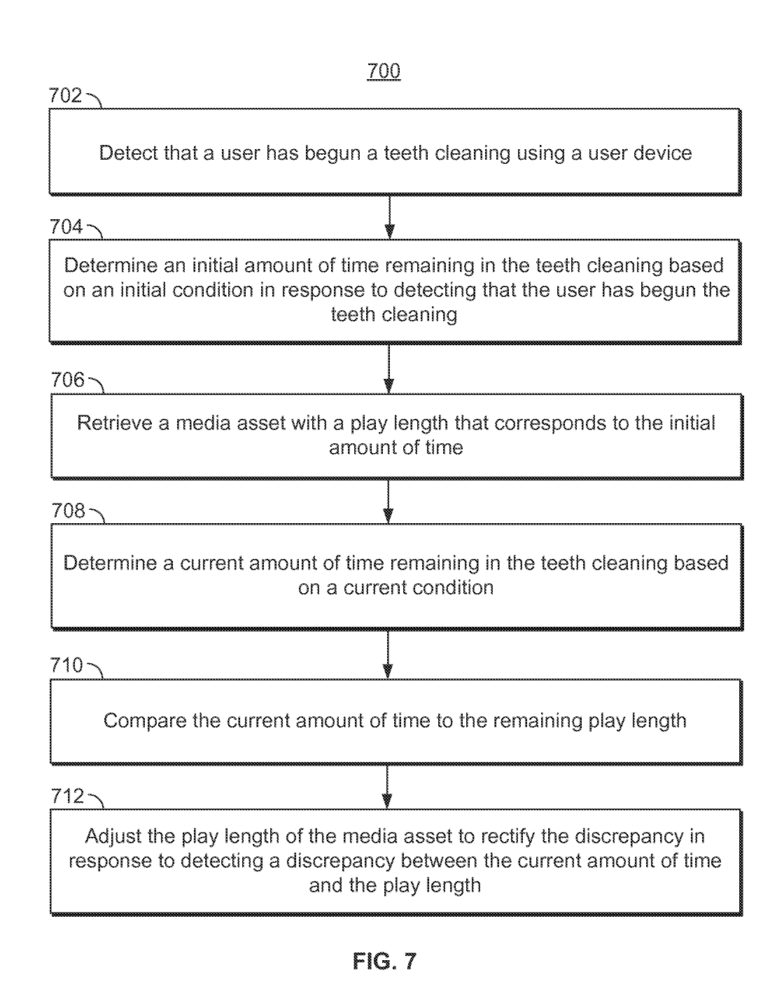

FIG. 6 is a flowchart of illustrative steps for adjusting the play length of a media asset associated with a current activity based on the length of time required to perform a previous activity. It should be noted that process 600 or any step thereof could be performed on, or provided by, any of the devices shown in FIGS. 3-4. For example, process 600 may be executed by control circuitry 304 (FIG. 3) as instructed by a media guidance application implemented on a user device (e.g., user equipment devices 402, 404, and/or 406 (FIG. 4)) in order to adjust the play length of a media asset based on a user's progress in an activity. In addition, one or more steps of process 600 may be incorporated into or combined with one or more steps of any other process or embodiment (e.g., as described in relation to FIGS. 7-9)).

At step 602, the media guidance application determines (e.g., via control circuitry 304 (FIG. 3)) a first activity and a second activity to be performed by a user in series. For example, the media guidance application may receive (e.g., via user input interface 310 (FIG. 3)) a user input indicating that during a certain time period (e.g., seven o'clock to eight o'clock) the user performs three different activities in series. For example, the media guidance application may receive information using an interface as shown in FIG. 2. Alternatively or additionally, the media guidance application may passively monitor (e.g., via detection module 316 (FIG. 3)) a user and generate a user profile of typical habits or routines of the user in order to determine what activities, and when, will be performed.

At step 604, the media guidance application determines (e.g., via control circuitry 304 (FIG. 3)) a first amount of time required to perform the first activity and a second amount of time to perform the second activity. For example, the media guidance application may cross-reference a database (e.g., located at storage 308 (FIG. 3) and/or any location accessible via communications network 414 (FIG. 4)) that lists approximate times for different activities to determine an approximate time for each of the first and second activities.

For example, the media guidance application may track user actions over a period of time to determine an average amount of time that any one activity requires. Additionally or alternatively, the media guidance application may receive information for the amount of time that one or more activities typically requires (e.g., from media content source 416 (FIG. 4), media guidance data source 418 (FIG. 4), and/or any location accessible vie communications network 414 (FIG. 4)). For example, the media guidance application may receive industrial reports or market research indicating the average amount of time that one or more users typically require to perform the one or more activities. Alternatively or additionally, the media guidance application may receive (e.g., via user input 310 (FIG. 3)) one or more user inputs indicating a schedule (e.g., the amount of time one or more activities as well as when each of the one or more activities begins).

At step 606, the media guidance application selects (e.g., via control circuitry 304 (FIG. 3)) a first media asset to present to the user while the user is performing the first activity and a second media asset to present to the user while the user is performing the second activity, in which a play length of the first media asset corresponds to the first amount of time and a play length of the second media asset corresponds to the second amount of time. For example, if the first activity (e.g., vacuuming the living room) requires ten minutes to perform, the media guidance application may select a media asset (e.g., a news clip) that last for ten minutes. Alternatively, the media guidance application may select several media assets that, when combined, last for ten minutes.

At step 608, in response to determining that the first activity has been completed, the media guidance application may compare (e.g., via control circuitry 304 (FIG. 3)) the first amount of time to an actual amount of time required to perform the first activity. For example, the media guidance application may monitor (e.g., via detection module 316 (FIG. 3)) user progress corresponding to the performance of the first activity to determine when the first activity has been completed.

For example, the media guidance application may compare the first amount of time (e.g., in a first unit of measure such as minutes) to an actual amount of time required to perform the first activity (e.g., in the first unit of measure such as minutes). Based on the comparison, the media guidance application may determine whether the first amount of time is greater than or less than the actual amount of time required to perform the first activity.

At step 610, in response to detecting a discrepancy between the first amount of time and the actual amount of time, the media guidance application may adjust (e.g., via control circuitry 304 (FIG. 3)) the play length of the second media asset to rectify the discrepancy. For example, if a user completed the first activity earlier than scheduled (e.g., as determined by detection module 316 (FIG. 3)), the media guidance application may give the user more time to perform the second activity. Accordingly, the media guidance application may adjust the play length of the second media asset by extending the play length of the second media asset to including additional content. In another example, if a user completed the first activity later than scheduled, the media guidance application may give the user less time to perform the second activity. Accordingly, the media guidance application may adjust the play length of the second media asset by decreasing the play length of the media asset by cropping a portion of media asset.

In some embodiments, the media guidance application may receive user input indicating criteria for select media assets. For example, the media guidance application may receive a user input selecting a subject matter for the first media asset and the second media asset and then search a media content source for media assets corresponding to the subject matter. In another example, the media guidance application may determine a first output format (e.g., video, audio, textual, etc.) for the first media asset and a second output format for the second media asset based on the activity being performed (e.g., whether or not a particular activity is conducive to a user consuming a video simultaneously). For example, the media guidance application may determine (e.g., via control circuitry 304 (FIG. 3)) that a particular output type is not conducive to, appropriate for, and/or corresponds to a user's preferences being consumed during particular activities. For example, while a text based media asset is conducive to being consumed by a user while the user is eating breakfast, the same text based media asset may not be conducive to being consumed while a user is showering. Likewise, while an audio based media asset may be conducive to being consumed by a user while the user is eating breakfast, the media guidance application may determine that a user would prefer to consume a video or text-based media asset during that activity. In response, the media guidance application may search a media content source for media assets corresponding to the first output format and the second output format.

In some embodiments, the media guidance application may reward a user for completing one or more activities early. For example, the media guidance application may provide the user with providing the user with additional content, an increased score, or a product offering in response to determining that the user has completed one or more activities earlier than scheduled. For example, a first user (e.g., a parent) may schedule a series of activities to be performed by a second user (e.g., a child). If the second user successfully performs each of the scheduled activities, the media guidance application may retrieve additional content and/or a product offering as a reward. Alternatively, the second user may be associated with a user profile that tracks the performance of the second user over time. Each time the user successfully performs (or fails to perform) an activity, the media guidance application may add (or subtract) points or adjust a score in the user profile. The media guidance application may then determine whether or not the current score of a user qualifies the user for one or more rewards.

It is contemplated that the steps or descriptions of FIG. 6 may be used with any other embodiment of this disclosure. In addition, the steps and descriptions described in relation to FIG. 6 may be done in alternative orders or in parallel to further the purposes of this disclosure. For example, each of these steps may be performed in any order or in parallel or substantially simultaneously to reduce lag or increase the speed of the system or method. Furthermore, it should be noted that any of the devices or equipment discussed in relation to FIGS. 3-4 could be used to perform one or more of the steps in FIG. 6.

FIG. 7 is a flowchart of illustrative steps for adjusting the play length of a media asset based on a user's progress in an activity. It should be noted that process 700 or any step thereof could be performed on, or provided by, any of the devices shown in FIGS. 3-4. For example, process 700 may be executed by control circuitry 304 (FIG. 3) as instructed by a media guidance application implemented on a user device (e.g., user equipment devices 402, 404, and/or 406 (FIG. 4)) in order to adjust the play length of a media asset based on a user's progress in an activity. In addition, one or more steps of process 700 may be incorporated into or combined with one or more steps of any other process or embodiment (e.g., as described in relation to FIGS. 6 and 8-9)).

At step 702, the media guidance application detects (e.g., via detection module 316) that a user has begun a teeth cleaning using a user device (e.g., an electric tooth brush). For example, the media guidance application may monitor (e.g., via detection module 316 (FIG. 3)) a user to determine when the user uses a specific user device (e.g., such as an electric tooth brush). Alternatively or additionally, the media guidance application may receive (e.g., via I/O path 302 (FIG. 3)) information that is transmitted from the user device (e.g., as detected by detection module 316 (FIG. 3) incorporated into the user device) that indicates that a user has begun an activity.

At step 704, the media guidance application may determine (e.g., via control circuitry 304 (FIG. 3)) an initial amount of time remaining in the teeth cleaning based on an initial condition in response to detecting that the user has begun the teeth cleaning. For example, based on one or more sensors (e.g., incorporated into, or accessible by detection module 316 (FIG. 3) in a user device) the user device may determine a total surface area that needs to be clean or an amount of dental plaque that needs to be removed and calculate an amount of time remaining in the teeth cleaning based that determination. Additionally or alternatively, the media guidance application may access (e.g., via control circuitry 304 (FIG. 3)) a user profile (e.g., located at storage 308 (FIG. 3) and/or any location accessible via communications network 414 (FIG. 4)) associated with the user that indicates a typical length of time required for the user to perform the teeth cleaning.

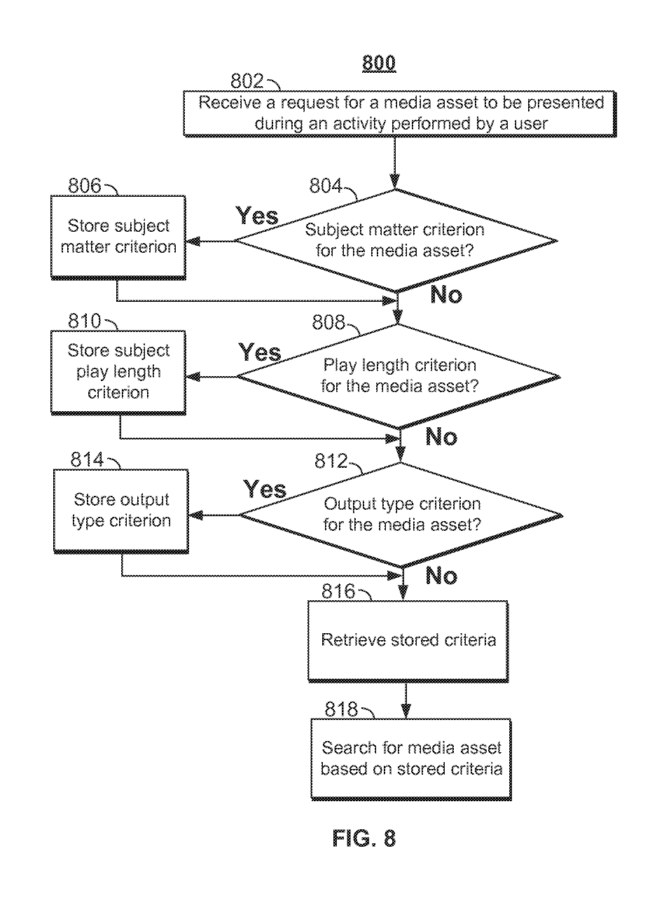

At step 706, the media guidance application retrieves a media asset with a play length that corresponds to the initial amount of time. For example, after determining how long a user is likely to take to perform the teeth cleaning, the media guidance application may search for a media asset with a play length that matches the determined length of time. For example, the media guidance application may access a database (e.g., located at storage 308 (FIG. 3), media content source 418 (FIG. 4), and/or any location accessible via communications network 414 (FIG. 4)). The media guidance application may filter the available media assets based on whether or not the play length of a media asset corresponds to the amount of time required to perform the teeth cleaning. In some embodiments, the media guidance application may select (e.g., via control circuitry 304 (FIG. 3)) a media asset based on one or more other criteria (e.g., as discussed in relation to FIG. 8)).

At step 708, the media guidance application determines (e.g., via control circuitry 304 (FIG. 3)) a current amount of time remaining in the teeth cleaning based on a current condition. For example, the user device (e.g., via a detection module incorporated into the user device) may periodically determine the total surface area of the teeth that still needs to be cleaned or determine an amount of dental plaque that still needs to be removed and calculate a new amount of time remaining in the teeth cleaning based the latest determination. In some embodiments, the conditions and requirements for conditions to be met (or not met) may be retrieved by the media guidance application from a database (e.g., located at storage 308 (FIG. 3) and/or any location accessible via communications network 414 (FIG. 4)). In some embodiments, the data in the database may be based on user inputs (e.g., via display 200 (FIG. 2)). Alternatively or additionally, the media guidance application may retrieve the conditions from a third party (e.g., dental association standards, dental records for the user, prescriptions for the user, etc.).

At step 710, the media guidance application compares (e.g., via control circuitry 304 (FIG. 3)) the current amount of time to the remaining play length. For example, the media guidance application may determine (e.g., via control circuitry 304 (FIG. 3)) whether the initial amount of time corresponds to the current amount of time in order to determine whether or not the user is on schedule (e.g., whether or not a current condition of the mouth of a user matches an approximated condition of the user based on the schedule).

At step 712, the media guidance application may then adjust (e.g., via control circuitry 304 (FIG. 3)) the play length of the media asset to rectify the discrepancy. For example, in response to determining (e.g., via control circuitry 304 (FIG. 3)) that the teeth cleaning will take longer (or shorter) than initially expected, the media guidance application may modify the length of time it takes for the user to consume a media asset that accompanies the activity. For example, the media guidance application may extend (e.g., via control circuitry 304 (FIG. 3)) the play length of the media asset by including additional content. In another example, the media guidance application may extend (e.g., via control circuitry 304 (FIG. 3)) the play length of the media asset by reducing a playback rate associated with the media asset. In another example, the media guidance application may decrease (e.g., via control circuitry 304 (FIG. 3)) the play length of the media asset by advancing a current point of playback in the media asset to a later point of playback in the media asset. In yet another example, the media guidance application may decrease (e.g., via control circuitry 304 (FIG. 3)) the play length of the media asset by cropping a portion of media asset.

In some embodiments, the media guidance application may additionally provide (e.g., via control circuitry 304 (FIG. 3)) user progress feedback to the user while the user is performing during the teeth cleaning. For example, the media guidance application may indicate (e.g., via control circuitry 304 (FIG. 3)) how much time remains for the scheduled activity. Alternatively or additionally, the media guidance application may provide (e.g., via control circuitry 304 (FIG. 3)) instructions associated with the teeth cleaning. For example, the media guidance application may instruct (e.g., via control circuitry 304 (FIG. 3)) the user as to particular teeth that need to be cleaned.

It is contemplated that the steps or descriptions of FIG. 7 may be used with any other embodiment of this disclosure. In addition, the steps and descriptions described in relation to FIG. 7 may be done in alternative orders or in parallel to further the purposes of this disclosure. For example, each of these steps may be performed in any order or in parallel or substantially simultaneously to reduce lag or increase the speed of the system or method. Furthermore, it should be noted that any of the devices or equipment discussed in relation to FIGS. 3-4 could be used to perform one or more of the steps in FIG. 7.

FIG. 8 is a flowchart of illustrative steps for searching for media assets based on particular criteria. It should be noted that process 800 or any step thereof could be performed on, or provided by, any of the devices shown in FIGS. 3-4. For example, process 800 may be executed by control circuitry 304 (FIG. 3) as instructed by a media guidance application implemented on a user device (e.g., user equipment devices 402, 404, and/or 406 (FIG. 4)) in order to search for media assets based on particular criteria. In addition, one or more steps of process 800 may be incorporated into or combined with one or more steps of any other process or embodiment (e.g., as described in relation to FIGS. 6-7 and 9)).

At step 802, the media guidance application receives a request for a media asset to be presented during an activity performed by a user. For example, the media guidance application may in response to determining (e.g., via detection module 316 (FIG. 3)) that a user is performing or is about to perform an activity may request a media asset. In another example, in response to a user request (e.g., received via use input interface 310 (FIG. 3)) for a media asset (e.g., in order to accompany an activity).

At step 804, the media guidance application determines (e.g., via control circuitry 304 (FIG. 3)) whether or not there is a subject matter criterion for selection of the media asset. For example, the media guidance application may reference settings (e.g., entered via display 200 (FIG. 2)) that indicate whether or not the user prefers for the media assets selected to accompany an activity should have a particular subject matter. In some embodiments, the subject matter selected may be specific to a particular activity. For example, performing a teeth cleaning may be associated with a particular genre.

If the media guidance application determines that there is a subject matter criterion for selection of the media asset, the media guidance application proceeds to step 806 and stores (e.g., at storage 308 (FIG. 3) and/or any location accessible via communications network 414 (FIG. 4)) the subject matter criterion, prior to proceeding to step 808. If the media guidance application determines that there is no subject matter criterion for the selection of the media asset, the media guidance application proceeds to step 808 directly.

At step 808, the media guidance application determines (e.g., via control circuitry 304 (FIG. 3)) whether or not there is a play length criterion for selection of the media asset. For example, the media guidance application may determine whether or not the selected media asset should have a particular play length. For example, the media guidance application may select (e.g., via control circuitry 304 (FIG. 3)) a media asset that has a play length that corresponds to the length of time required to perform a particular activity. Alternatively, the media guidance application may determine (e.g., via control circuitry 304 (FIG. 3)) that there is no play length criterion. For example, the media guidance application may select a media asset irrespective of whether or not the media asset's play length corresponds to a particular activity. In such cases, the media guidance may segregate the media asset into different segments, in which each segment corresponds to the time required to perform an activity. In some embodiments, the subject matter selected may be specific to a particular activity. For example, performing a teeth cleaning may be associated with a particular genre.

If the media guidance application determines that there is a subject matter criterion for selection of the media asset, the media guidance application proceeds to step 810 and stores (e.g., at storage 308 (FIG. 3) and/or any location accessible via communications network 414 (FIG. 4)) the play length criterion, prior to proceeding to step 812. If the media guidance application determines that there is no play length criterion for the selection of the media asset, the media guidance application proceeds to step 812 directly.

At step 812, the media guidance application determines (e.g., via control circuitry 304 (FIG. 3)) whether or not there is a output type criterion for selection of the media asset. For example, the media guidance application may reference settings (e.g., entered via display 200 (FIG. 2)) that indicate whether or not the user prefers for the media assets selected to accompany an activity should have a particular output type. Alternatively or additionally, the media guidance application may determine (e.g., via control circuitry 304 (FIG. 3)) a first output format (e.g., video, audio, textual, etc.) for a first media asset and a second output format for a second media asset based on the activity being performed (e.g., whether or not a particular activity is conducive to a user consuming a video simultaneously).

For example, the media guidance application may determine (e.g., via control circuitry 304 (FIG. 3)) that a particular output type is not conducive to, appropriate for, and/or corresponds to a user's preferences to being consumed during particular activities. For example, while a text based media asset is conducive to being consumed by a user while the user is riding on the subway to work, the same text based media asset may not be conducive to being consumed while a user is driving to work. Likewise, while an audio based media asset may be conducive to being consumed by a user while the user is exercising, the media guidance application may determine that a user would prefer to consume a video or text-based media asset during that activity.

If the media guidance application determines that there is an output type criterion for selection of the media asset, the media guidance application proceeds to step 814 and stores (e.g., at storage 308 (FIG. 3) and/or any location accessible via communications network 414 (FIG. 4)) the subject matter criterion, prior to proceeding to step 816. If the media guidance application determines that there is no subject matter criterion for the selection of the media asset, the media guidance application proceeds to step 816 directly.

At step 810, the media guidance application retrieves the stored criteria. For example, if the media guidance application determines to select a media asset based on the subject matter, play length, and/or output type, the media guidance application retrieves the criterion or criteria for each. At step 818, the media guidance application searches one or more content sources (e.g., storage 308 (FIG. 3)), media content source 416 (FIG. 4), media guidance data source 418 (FIG. 4), an/or any location accessible via communications network 414 (FIG. 4)).

It is contemplated that the steps or descriptions of FIG. 8 may be used with any other embodiment of this disclosure. In addition, the steps and descriptions described in relation to FIG. 8 may be done in alternative orders or in parallel to further the purposes of this disclosure. For example, each of these steps may be performed in any order or in parallel or substantially simultaneously to reduce lag or increase the speed of the system or method. Furthermore, it should be noted that any of the devices or equipment discussed in relation to FIGS. 3-4 could be used to perform one or more of the steps in FIG. 8.

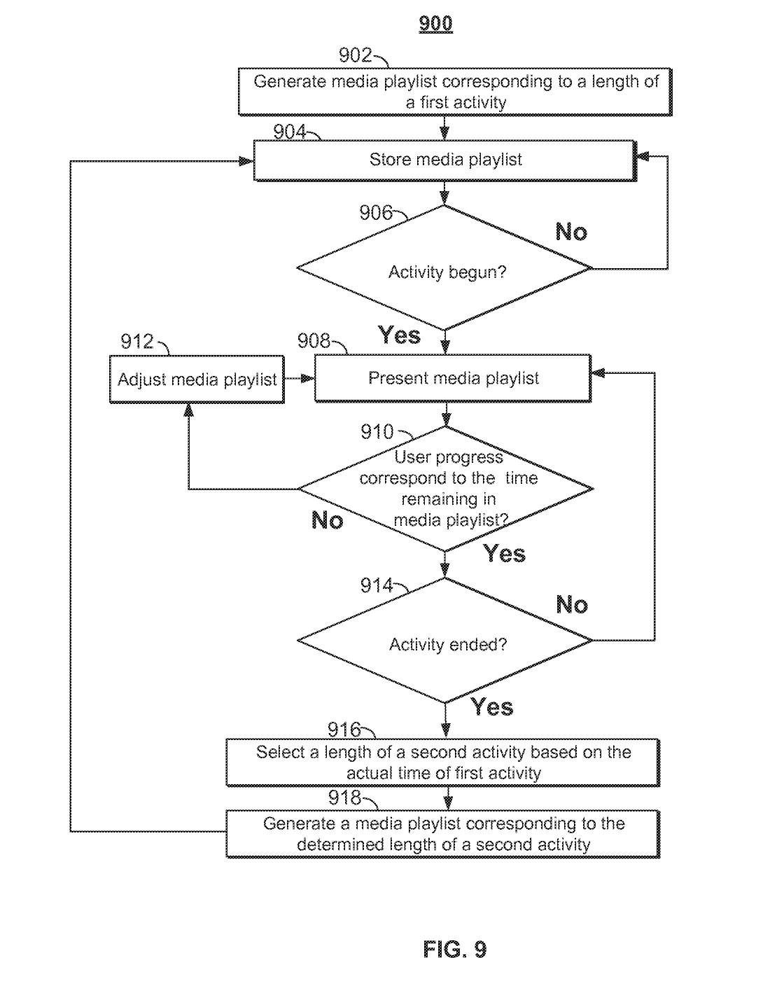

FIG. 9 is a flowchart of illustrative steps for adjusting a playlist based on the length of time required to perform an activity. It should be noted that process 900 or any step thereof could be performed on, or provided by, any of the devices shown in FIGS. 3-4. For example, process 900 may be executed by control circuitry 304 (FIG. 3) as instructed by a media guidance application implemented on a user device (e.g., user equipment devices 402, 404, and/or 406 (FIG. 4)) in order to adjust a playlist based on the length of time required to perform an activity. In addition, one or more steps of process 900 may be incorporated into or combined with one or more steps of any other process or embodiment (e.g., as described in relation to FIGS. 6-8)).

At step 902, the media guidance application generates a media playlist corresponds to a length of a first activity. For example, as discussed above in relation to step 604 (FIG. 6) the media guidance application may determine (e.g., via control circuitry 304 (FIG. 3)) an amount of time required to perform an activity. For example, the media guidance application may cross-reference a database (e.g., located at storage 308 (FIG. 3) and/or any location accessible via communications network 414 (FIG. 4)) that lists approximate times for different activities to determine an approximate time for each of the activity. In response, the media guidance application may select (e.g., as discussed in relation to FIG. 8) a media asset or media assets for inclusion in a playlist to be presented while the user is performing the activity.

At step 904, the media guidance application stores the media playlist. For example, the media guidance application may store (e.g., in storage 308 (FIG. 3) and/or any location accessible via communications network 414 (FIG. 4)) the media playlist for presentation when a user begins an activity. For example, the media guidance application may store numerous playlist associated with one more users and/or one or more activities.

At step 906, the media guidance application determines (e.g., via control circuitry 304 (FIG. 3)) whether or not an activity has begun. For example, the media guidance application may incorporate or have access to a detection module (e.g., detection module 316 (FIG. 3)) that alerts the media guidance application that a user has begun an activity. Alternatively or additionally, the media guidance application may receive (e.g., via I/O path 302 (FIG. 3)) information from another device (e.g., user device 506 (FIG. 5)) indicating that a user has begun an activity.

If the media guidance application determines that an activity has begin, the media guidance application proceeds to step 908. If the media guidance application determines that an activity has not yet begun, the media guidance application returns to step 904. At step 908, the media guidance application presents (e.g., via control circuitry 304 (FIG. 3)) the media playlist. For example, the media guidance application may (e.g., via control circuitry 304 (FIG. 3)) generate for display the media playlist (e.g., featuring one or more media assets) on user equipment device 402, 404, and/or 406 (FIG. 4).

At step 910, the media guidance application determines whether or not the user progress corresponds to the time remaining in the media playlist. For example, the media guidance application may determine (e.g., via detection module 316 (FIG. 3)) a point of user progress in the performance of the activity. In some embodiments, the point of user progress may correspond to a particular condition. Furthermore, the media guidance application may determine (e.g., via control circuitry 304 (FIG. 3)) whether or not the current point of progress corresponds to the amount of time remaining in the media playlist.

For example, the media guidance application may cross-reference (e.g., via control circuitry 304 (FIG. 3)) the current point of progress and/or the current condition in a database listing points of progress and/or conditions in an activity that corresponds to amounts of time remaining. The media guidance application may input (e.g., via control circuitry 304 (FIG. (3)) the current point of progress and/or the current condition in the database and receive an output corresponding to a scheduled amount of time that should be remaining. The media guidance application may then compare (e.g., via control circuitry 304 (FIG. (3)) the output and actual amount of time remaining to determine whether the user is on schedule.

If the media guidance application determines that the user progress corresponds to the amount of time remaining in the media playlist, the media guidance application proceeds to step 914. If the media guidance application determines that the user progress does not correspond to the amount of time remaining in the media playlist, the media guidance application proceeds to step 912. At step 912, the media guidance application adjusts the media playlist and returns to step 908. For example, in response to determining (e.g., via control circuitry 304 (FIG. 3)) that an activity (e.g., teeth cleaning) will take longer (or shorter) than initially expected, the media guidance application may modify the length of time it takes for the user to consume a media playlist that accompanies the activity. For example, the media guidance application may extend (e.g., via control circuitry 304 (FIG. 3)) the play length of the media playlist by including additional content (e.g., additional media assets or portions of media assets). In another example, the media guidance application may extend (e.g., via control circuitry 304 (FIG. 3)) the play length of the media playlist by reducing a playback rate associated with the media asset.

In another example, the media guidance application may decrease (e.g., via control circuitry 304 (FIG. 3)) the play length of the media playlist by advancing a current point of playback in the media asset to a later point of playback in the media asset. In yet another example, the media guidance application may decrease (e.g., via control circuitry 304 (FIG. 3)) the play length of the media playlist by cropping a portion of the media asset.

At step 914, the media guidance application determines (e.g., via control circuitry 304 (FIG. 3)) whether or not an activity has ended. For example, the media guidance application may incorporate or have access to a detection module (e.g., detection module 316 (FIG. 3)) that indicates to the media guidance application when an activity has been completed (or the performance of the activity has been stopped). Alternatively or additionally, the media guidance application may receive (e.g., via I/O path 302 (FIG. 3)) information from a user device (e.g., user device 506 (FIG. 5)) that indicates the status of an activity.

If the media guidance application determines (e.g., via control circuitry 304 (FIG. 3)) that the activity has not ended, the media guidance application returns to step 908. If the media guidance application determines that the activity has ended, the media guidance application proceeds to step 916.

At step 916, the media guidance application determines (e.g., via control circuitry 304 (FIG. 3)) a length of a second activity based on the actual time of the first activity. For example, if the user took longer than expected to perform the first activity, the media guidance application may schedule less time for performance of the second activity. Alternatively, if the user took less time than expected to perform the first activity, the media guidance application may schedule more time for performance of the second activity. For example, in response to detecting that a user completed a first activity (e.g., a chore) quickly, the media guidance application may allot more time for a second activity (e.g., a reward).

At step 918, the media guidance application generates a media playlist corresponding to the determined length of a second activity and returns to step 904. For example, as discussed above, the media guidance application may determine (e.g., via control circuitry 304 (FIG. 3)) an amount of time required to perform the second activity. For example, the media guidance application may cross-reference a database (e.g., located at storage 308 (FIG. 3) and/or any location accessible via communications network 414 (FIG. 4)) that lists approximate times for different activities, including the second activity, to determine an approximate time for second activity. In response, the media guidance application may select (e.g., as discussed in relation to FIG. 8) a media asset or media assets for inclusion in a playlist to be presented while the user is performing the second activity.

It is contemplated that the steps or descriptions of FIG. 9 may be used with any other embodiment of this disclosure. In addition, the steps and descriptions described in relation to FIG. 9 may be done in alternative orders or in parallel to further the purposes of this disclosure. For example, each of these steps may be performed in any order or in parallel or substantially simultaneously to reduce lag or increase the speed of the system or method. Furthermore, it should be noted that any of the devices or equipment discussed in relation to FIGS. 3-4 could be used to perform one or more of the steps in FIG. 9.

The above-described embodiments of the present disclosure are presented for purposes of illustration and not of limitation, and the present disclosure is limited only by the claims that follow. Furthermore, it should be noted that the features and limitations described in any one embodiment may be applied to any other embodiment herein, and flowcharts or examples relating to one embodiment may be combined with any other embodiment in a suitable manner, done in different orders, or done in parallel. In addition, the systems and methods described herein may be performed in real time. It should also be noted that the systems and/or methods described above may be applied to, or used in accordance with, other systems and/or methods.

1. A method for modifying a media asset, the method comprising:
detecting that a user has begun a teeth cleaning using a user device;
in response to detecting that the user has begun the teeth cleaning, determining an initial amount of time remaining in the teeth cleaning based on an initial condition;
retrieving a media asset with a play length that corresponds to the initial amount of time;
determining a current amount of time remaining in the teeth cleaning based on a current condition;
comparing the current amount of time to the remaining play length; and
in response to detecting a discrepancy between the current amount of time and the play length, adjusting the play length of the media asset to rectify the discrepancy.

2. The method of claim 1, wherein adjusting the play length of the media asset includes extending the play length of the media asset by including additional content.

3. The method of claim 1, wherein adjusting the play length of the media asset includes extending the play length of the media asset by reducing a playback rate associated with the media asset.

4. The method of claim 1, wherein adjusting the play length of the media asset includes decreasing the play length of the media asset by advancing a current point of playback in the media asset to a later point of playback in the media asset.

5. The method of claim 1, wherein adjusting the play length of the media asset includes decreasing the play length of the media asset by cropping a portion of media asset.

6. The method of claim 1, further comprising generating for display an indication of the current condition.

7. The method of claim 1, further comprising generating for display instructions associated with the teeth cleaning.

8. The method of claim 1, wherein the user device is an electric toothbrush.

9. The method of claim 1, wherein the media asset is generated for display on a display device in response to information transmitted from the user device.

10. The method of claim 1, wherein the current condition includes an amount of tooth surface area remaining to be cleaned or an amount of dental plaque needing to be removed.

11. A system for modifying a media asset, the system comprising:
detection circuitry configured to detect that a user has begun a teeth cleaning using a user device; and
control circuitry configured to:
in response to detecting that the user has begun the teeth cleaning, determine an initial amount of time remaining in the teeth cleaning based on an initial condition;
retrieve a media asset with a play length that corresponds to the initial amount of time;
determine a current amount of time remaining in the teeth cleaning based on a current condition;
compare the current amount of time to the remaining play length; and
in response to detecting a discrepancy between the current amount of time and the play length, adjust the play length of the media asset to rectify the discrepancy.

12. The system of claim 11, wherein the control circuitry configured to adjust the play length of the media asset is further configured to extend the play length of the media asset by including additional content.

13. The system of claim 11, wherein the control circuitry configured to adjust the play length of the media asset is further configured to extend the play length of the media asset by reducing a playback rate associated with the media asset.

14. The system of claim 11, wherein the control circuitry configured to adjust the play length of the media asset is further configured to decrease the play length of the media asset by advancing a current point of playback in the media asset to a later point of playback in the media asset.

15. The system of claim 11, wherein the control circuitry configured to adjust the play length of the media asset is further configured to decrease the play length of the media asset by cropping a portion of media asset.

16. The system of claim 11, wherein the control circuitry is further configured to generate for display an indication of the current condition.

17. The system of claim 11, wherein the control circuitry is further configured to generate for display instructions associated with the teeth cleaning.

18. The system of claim 11, wherein the user device is an electric toothbrush.

19. The system of claim 11, wherein the media asset is generated for display on a display device in response to information transmitted from the user device.

20. The system of claim 11, wherein the current condition includes an amount of tooth surface area remaining to be cleaned or an amount of dental plaque needing to be removed.

21-50. (canceled)
